(19) United States
(12) Patent Application Publication
(10) Pub. No.: US 2005/0234650 A1
(43) Pub. Date: Oct. 20, 2005

(54) Method and system for normalization of microarray data

(76) Inventors: Zohar Yakhini, Ramat Hasharon (IL); Amir Ben-Dor, Bellevue, WA (US); Nicholas M. Sampas, San Jose, CA (US)

Correspondence Address: AGILENT TECHNOLOGIES, INC.;Legal Department, DL429, Intellectual Property Administration, P.O. Box 7599, Loveland, CO, 80537-0599, US

(21) Appl. No.: 10/825,893

(22) Filed: Apr. 16, 2004

(51) Int. Cl. G06F 19/00; G01N 33/48; G01N 33/50

(52) U.S. Cl. 702/19; 702/20

(57) ABSTRACT

One embodiment of the present invention provides a method and system for selecting a subset of normalization features, or biomolecule probes, from multiple data sets generated from a single microarray and from multiple data sets generated from multiple microarrays. In this embodiment, the signal intensities corresponding to common features within the data sets are viewed as generating a distribution of features within an n-dimensional signal-intensity distribution. One or more order-preserving sequences of features within the n-dimensional signal-intensity distribution are determined using an efficient, two-pass-per-dimension method. Normalizing features then selected from the one or more order-preserving sequences. Normalizing data points from generalized data sets may be obtained by using embodiments of the present invention.

### TECHNICAL FIELD

The present invention is related to processing of molecular-array data and, in particular, to selection of the normalization probes or features for the normalization of multiple data sets obtained from a single microarray, or from multiple data sets obtained from multiple microarrays.

### BACKGROUND OF THE INVENTION

The present invention is related to normalization of data sets obtained by scanning a microarray at different optical frequencies, in the case of chromophore-labeled target molecules, or at different radioactive emission energies, in the case of isotopically labeled target molecules, and to normalization of data sets obtained by scanning two or more microarrays at one or more optical frequencies or radioactive emission energies. A general background of microarray technology is first provided, in this section, to facilitate discussion of the scanning techniques described in following sections. Microarrays are also referred to as “molecular arrays” and simply as “arrays” in the literature. Microarrays are not arbitrary regular patterns of molecules, such as occur on the faces of crystalline materials, but, as the following discussion shows, are manufactured articles specifically designed for analysis of solutions of compounds of chemical, biochemical, biomedical, and other interests.

Array technologies have gained prominence in biological research and are likely to become important and widely used diagnostic tools in the healthcare industry. Currently, microarray techniques are most often used to determine the concentrations of particular nucleic-acid polymers in complex sample solutions, or the presence or absense of certain chemical species, such as mutant or wild-type forms of DNA or of genes or messages associated with a disease state or phenotypic condition. Microarray-based analytical techniques are not, however, restricted to analysis of nucleic acid solutions, but may be employed to analyze complex solutions of any type of molecule that can be optically or radiometrically scanned or read and that can bind with high specificity to complementary molecules synthesized within, or bound to, discrete features on the surface of an array. Because arrays are widely used for analysis of nucleic acid samples, the following background information on arrays is introduced in the context of analysis of nucleic acid solutions following a brief background of nucleic acid chemistry.

Deoxyribonucleic acid (“DNA”) and ribonucleic acid (“RNA”) are linear polymers, each synthesized from four different types of subunit molecules. The subunit molecules for DNA include: (1) deoxy-adenosine, abbreviated “A,” a purine nucleoside; (2) deoxy-thymidine, abbreviated “T,” a pyrimidine nucleoside; (3) deoxy-cytosine, abbreviated “C,” a pyrimidine nucleoside; and (4) deoxy-guanosine, abbreviated “G,” a purine nucleoside. FIG. 1 illustrates a short DNA polymer 100, called an oligomer, composed of the following subunits: (1) deoxy-adenosine 102; (2) deoxy-thymidine 104; (3) deoxy-cytosine 106; and (4) deoxy-guanosine 108. When phosphorylated, subunits of DNA and RNA molecules are called “nucleotides” and are linked together through phosphodiester bonds 110-115 to form DNA and RNA polymers. A linear DNA molecule, such as the oligomer shown in FIG. 1, has a 5′ end 118 and a 3′ end 120. A DNA polymer can be chemically characterized by writing, in sequence from the 5′ end to the 3′ end, the single letter abbreviations for the nucleotide subunits that together compose the DNA polymer. For example, the oligomer 100 shown in FIG. 1 can be chemically represented as “ATCG.” A DNA nucleotide comprises a purine or pyrimidine base (e.g. adenine 122 of the deoxy-adenylate nucleotide 102), a deoxy-ribose sugar (e.g. deoxy-ribose 124 of the deoxy-adenylate nucleotide 102), and a phosphate group (e.g. phosphate 126) that links one nucleotide to another nucleotide in the DNA polymer.

The DNA polymers that contain the organization information for living organisms occur in the nuclei of cells in pairs, forming double-stranded DNA helixes. One polymer of the pair is laid out in a 5′ to 3′ direction, and the other polymer of the pair is laid out in a 3′ to 5′ direction. The two DNA polymers in a double-stranded DNA helix are therefore described as being anti-parallel. The two DNA polymers, or strands, within a double-stranded DNA helix are bound to each other through attractive forces including hydrophobic interactions between stacked purine and pyrimidine bases and hydrogen bonding between purine and pyrimidine bases, the attractive forces emphasized by conformational constraints of DNA polymers. Because of a number of chemical and topographic constraints, double-stranded DNA helices are most stable when deoxy-adenylate subunits of one strand hydrogen bond to deoxy-thymidylate subunits of the other strand, and deoxy-guanylate subunits of one strand hydrogen bond to corresponding deoxy-cytidilate subunits of the other strand.

FIGS. 2A-B illustrates the hydrogen bonding between the purine and pyrimidine bases of two anti-parallel DNA strands. AT and GC base pairs, illustrated in FIGS. 2A-B, are known as Watson-Crick (“WC”) base pairs. Two DNA strands linked together by hydrogen bonds forms the familiar helix structure of a double-stranded DNA helix. FIG. 3 illustrates a short section of a DNA double helix 300 comprising a first strand 302 and a second, anti-parallel strand 304.

Double-stranded DNA may be denatured, or converted into single stranded DNA, by changing the ionic strength of the solution containing the double-stranded DNA or by raising the temperature of the solution. Single-stranded DNA polymers may be renatured, or converted back into DNA duplexes, by reversing the denaturing conditions, for example by lowering the temperature of the solution containing complementary single-stranded DNA polymers. During renaturing or hybridization, complementary bases of anti-parallel DNA strands form WC base pairs in a cooperative fashion, leading to reannealing of the DNA duplex.

The ability to denature and renature double-stranded DNA has led to the development of many extremely powerful and discriminating assay technologies for identifying the presence of DNA and RNA polymers having particular base sequences or containing particular base subsequences within complex mixtures of different nucleic acid polymers, other biopolymers, and inorganic and organic chemical compounds. One such methodology is the array-based hybridization assay. FIGS. 4-7 illustrate the principle of the array-based hybridization assay. An array (402 in FIG. 4) comprises a substrate upon which a regular pattern of features is prepared by various manufacturing processes. The array 402 in FIG. 4, and in subsequent FIGS. 5-7, has a grid-like 2-dimensional pattern of square features, such as feature 404 shown in the upper left-hand corner of the array. Each feature of the array contains a large number of identical oligonucleotides covalently bound to the surface of the feature. These bound oligonucleotides are known as probes. In general, chemically distinct probes are bound to the different features of an array, so that each feature corresponds to a particular nucleotide sequence. In FIGS. 4-6, the principle of array-based hybridization assays is illustrated with respect to the single feature 404 to which a number of identical probes 405-409 are bound. In practice, each feature of the array contains a high density of such probes but, for the sake of clarity, only a subset of these are shown in FIGS. 4-6.

Once an array has been prepared, the array may be exposed to a sample solution of target DNA or RNA molecules (410-413 in FIG. 4) labeled with fluorophores, chemiluminescent compounds, or radioactive atoms 415-418. Labeled target DNA or RNA hybridizes through base pairing interactions to the complementary probe DNA, synthesized on the surface of the array. FIG. 5 shows a number of such target molecules 502-504 hybridized to complementary probes 505-507, which are in turn bound to the surface of the array 402. Targets, such as labeled DNA molecules 508 and 509, that do not contains nucleotide sequences complementary to any of the probes bound to array surface do not hybridize to generate stable duplexes and, as a result, tend to remain in solution. The sample solution is then rinsed from the surface of the array, washing away any unbound-labeled DNA molecules. In other embodiments, unlabeled target sample is allowed to hybridize with the array first. Typically, such a target sample has been modified with a chemical moiety that will react with a second chemical moiety in subsequent steps. Then, either before or after a wash step, a solution containing the second chemical moiety bound to a label is reacted with the target on the array. After washing, the array is ready for data acquisition by scanning or reading. Biotin and avidin represent an example of a pair of chemical moieties that can be utilized for such steps.

Finally, as shown in FIG. 6, the bound labeled DNA molecules are detected via optical or radiometric scanning or reading. Optical scanning and reading both involve exciting labels of bound labeled DNA molecules with electromagnetic radiation of appropriate frequency and detecting fluorescent emissions from the labels, or detecting light emitted from chemiluminescent labels. When radioisotope labels are employed, radiometric scanning or reading can be used to detect the signal emitted from the hybridized features. Additional types of signals are also possible, including electrical signals generated by electrical properties of bound target molecules, magnetic properties of bound target molecules, and other such physical properties of bound target molecules that can produce a detectable signal. Optical, radiometric, or other types of scanning and reading produce an analog or digital representation of the array as shown in FIG. 7, with features to which labeled target molecules are hybridized similar to 706 optically or digitally differentiated from those features to which no labeled DNA molecules are bound. In other words, the analog or digital representation of a scanned array displays positive signals for features to which labeled DNA molecules are hybridized and displays negative features to which no, or an undetectably small number of, labeled DNA molecules are bound. Features displaying positive signals in the analog or digital representation indicate the presence of DNA molecules with complementary nucleotide sequences in the original sample solution. Moreover, the signal intensity produced by a feature is generally related to the amount of labeled DNA bound to the feature, in turn related to the concentration, in the sample to which the array was exposed, of labeled DNA complementary to the oligonucleotide within the feature.

One, two, or more than two data subsets within a data set can be obtained from a single microarray by scanning or reading the microarray for one, two or more than two types of signals. Two or more data subsets can also be obtained by combining data from two different arrays. When optical scanning or reading is used to detect fluorescent or chemiluminescent emission from chromophore labels, a first set of signals, or data subset, may be generated by scanning or reading the microarray at a first optical wavelength, a second set of signals, or data subset, may be generated by scanning or reading the microarray at a second optical wavelength, and additional sets of signals may be generated by scanning or reading the molecular at additional optical wavelengths. Different signals may be obtained from a microarray by radiometric scanning or reading to detect radioactive emissions at one, two, or more than two different energy levels. Target molecules may be labeled with either a first chromophore that emits light at a first wavelength, or a second chromophore that emits light at a second wavelength. Following hybridization, the microarray can be scanned or read at the first wavelength to detect target molecules, labeled with the first chromophore, hybridized to features of the microarray, and can then be scanned or read at the second wavelength to detect target molecules, labeled with the second chromophore, hybridized to the features of the microarray. In one common microarray system, the first chromophore emits light at a near infrared wavelength, and the second chromophore emits light at a yellow visible-light wavelength, although these two chromophores, and corresponding signals, are referred to as “red” and “green.” The data set obtained from scanning or reading the microarray at the red wavelength is referred to as the “red signal,” and the data set obtained from scanning or reading the microarray at the green wavelength is referred to as the “green signal.” While it is common to use one or two different chromophores, it is possible to use one, three, four, or more than four different chromophores and to scan or read a microarray at one, three, four, or more than four wavelengths to produce one, three, four, or more than four data sets. With the use of quantum-dot dye particles, the emission is tunable by suitable engineering of the quantum-dot dyr particles, and a fairly large set of such quantum-dot dye particles can be excited with a single-color, single-laser-based excitation.

Thus, multiple data sets may be obtained from a single microarray, and multiple microarrays can generate multiple sets of data sets. These data sets have different meanings, depending on the different types of experiments in which the microarrays are exposed to target-molecule-containing solutions. Frequently, data sets scanned from multiple microarrays are experimentally related, and data sets scanned at different optical frequencies from a single microarray are commonly related to one another. However, in order to meaningfully analyze and compare multiple data sets, the multiple data sets need to be normalized with respect to one another.

FIG. 8 illustrates a data normalization problem for multiple data sets scanned from a single microarray. In FIG. 8, a single microarray 802 containing oligonucleotide gene probes is scanned at a first optical wavelength λ₁ to generate a first data set 804 and is scanned at a second optical wavelength λ₂ to generate a second data set 805. In FIG. 8, the data sets are shown as plots of signal intensities scanned from features. In each plot, the vertical axis, such as vertical axis 806, corresponds to intensity of a feature, and the horizontal axis, such as horizontal axis 808, corresponds to the index or position of the feature within the microarray. The data shown in FIG. 8 might be generated, for example, in an experiment where the target molecules labeled with a chromophore that emits light at wavelength λ₁ are hybridized to the microarray in an experiment in which the microarray is exposed to a sample solution collected from one or more organisms in a normal, unperturbed state. The second data set results from scanning of the microarray for target molecules, labeled with a chromophore that emits light at wavelength λ₂, present in a sample solution collected from one or more organisms in a diseased state, or following exposure to a toxic agent, an infectious particle, or to other chemical or biological insults. In such experiments, the experimenter is typically searching for some small subset of genes with increased or decreased expression levels in the sample collected following exposure of the organism or organisms to biochemical reagents, changes in environment, or to other such perturbations or changes in state. Many, if not most, of the measured gene-expression levels may not appreciably change, but those genes related to toxin-degradation pathways, infection-fighting systems, and other such logical subcomponents of an organism may fluctuate markedly with respect to their levels in the unperturbed state.

Comparing the two data sets 804 and 805 in FIG. 8, it is apparent that the relative intensities of the majority of the features are unchanged, with some features, shown in data set 805 by dashed arrows, such as dashed arrow 810, have relative intensities markedly different from those of the corresponding features in the unperturbed data set 804. Note, however, that the intensities of all features in data set 805 are shifted upward by a constant intensity value 812. The normalization problem, in this case, is to determine the value of this shift, so that the constant shift value can be subtracted from the intensities in data set 805 in order to place both data sets 804 and 805 onto a common, normalized intensity scale. Only by first normalizing data can the features with relative intensity changes be easily and quantitatively determined.

FIG. 9 illustrates a data normalization problem for multiple data sets scanned from two different microarrays. As shown on FIG. 9, two data sets 914 and 915 scanned from two microarrays 916 and 918, respectively, at a single wavelength λ₁ may also show a general constant shift in intensity 920 that must be determined in order to place both data sets on a common intensity scale. By converting to a common scale, it is possible to determine which features, such as feature 922, have different relative intensities in the two data sets and that thus indicate different gene expression levels in the two organisms or groups of organisms from which sample solutions were prepared for exposing microarrays 916 and 918.

FIGS. 8-9 illustrate a relatively simple normalization problem, where intensities are shifted by a constant factor. In real-life experiments, intensity shifts may not be constant, but may depend on the intensity values of the signals, on the identities of the features from which the signals are produced, and may be modeled by linear or more complex, nonlinear relationships.

There are many proposed techniques for carrying out normalization. A significant subset of these techniques relies on identifying a subset of features common to all data sets that are being normalized and that appear to exhibit little or no relative intensity changes among the data sets. For example, in gene-expression experiments, a subset of gene probes, or features, desirable for normalization would be features intended to bind to so-called “housekeeping genes,” genes whose expression levels appear to be generally unaffected over the course of the experiments from which the data sets sought to be normalized were generated. Finding such desirable subsets of features for normalization purposes is not, however, straightforward. The examples in FIGS. 8-9 are simple and exhibit a constant intensity shift. In real-life data sets, there may be thousands or tens of thousands of data points, and there may be many sources of signal variation between the intensities of corresponding features in each data set. These sources of signal-intensity variation may include experimental error, instrumental noise, non-uniform probe-molecule deposition, contamination, non-uniform labeling of target molecules, re-absorption of light emitted from chromophores, and many other such factors. These sources of intensity variation contribute to a general variation in feature intensities between data sets unrelated to the variations in signal strength and instrument sensitivity that are sought to be controlled by normalization. Determining a subset of features with relatively unchanged relative intensities requires somehow choosing a subset of common features within a distribution of feature intensities that appear to be expressed at relatively constant levels.

Because of the large number of individual data points within common data sets generated from microarrays, complex computational techniques applied to the data sets may suffer from well-known combinatorial explosion problems. Because data normalization can profoundly influence experimental conclusions drawn from normalized data sets, particularly conclusions based partially or wholly on relatively weak signals, reliability, repeatability, and accuracy can all be critically important. Designers, manufacturers, and users of microarrays have therefore recognized a need for a computationally efficient, reliable, and accurate technique for choosing a subset of feature intensities common to multiple experimentally related data sets that can be used to normalize the data sets one to another.

### SUMMARY OF THE INVENTION

One embodiment of the present invention provides a method and system for selecting a subset of normalization features, or biomolecule probes, from multiple data sets generated from a single microarray and from multiple data sets generated from multiple microarrays. In this embodiment, the signal intensities corresponding to common features within the data sets are viewed as generating a distribution of features within an n-dimensional signal-intensity distribution. One or more order-preserving sequences of features within the n-dimensional signal-intensity distribution are determined using an efficient, two-pass-per-dimension method. Normalizing features then selected from the one or more order-preserving sequences. Normalizing data points from generalized data sets may be obtained by using embodiments of the present invention.

### BRIEF DESCRIPTION OF THE DRAWINGS

FIG. 1 illustrates a short DNA polymer 100, called an oligomer, composed of the following subunits: (1) deoxy-adenosine 102; (2) deoxy-thymidine 104; (3) deoxy-cytosine 106; and (4) deoxy-guanosine 108.

FIGS. 2A-B illustrate the hydrogen bonding between the purine and pyrimidine bases of two anti-parallel DNA strands.

FIG. 3 illustrates a short section of a DNA double helix 300 comprising a first strand 302 and a second, anti-parallel strand 304.

FIGS. 4-7 illustrate the principle of the array-based hybridization assay.

FIG. 8 illustrates a data normalization problem for multiple data sets scanned from a single microarray.

FIG. 9 illustrates a data normalization problem for multiple data sets scanned from a two different microarrays.

FIGS. 10A-D illustrate a two-dimensional LOPS.

FIGS. 11A-C illustrate a LOPS within a three-dimensional distribution of data points.

FIG. 12 illustrates a distribution of the data points within four data sets for a simple example used to illustrate the computational technique that constitutes a portion of one embodiment of the present invention.

FIG. 13 is a table containing the data plotted in FIG. 12.

FIGS. 14A-K illustrate computation of a set of points coincident with longest-order-preserving sequences within the data-point shown in FIG. 12 and plotted in FIG. 13.

FIGS. 15A-B illustrate a relaxed comparison in which only three of the four values of a first data point must exceed the corresponding values of a second data point in order for the first data point to be considered to be greater than the second data point.

FIGS. 16A-D illustrate application of a more sophisticated method for producing an almost-LOPS set from the four-dimensional data-point distribution shown in FIG. 12.

FIGS. 17A-B illustrate the three highest almost-LOPS data points graphed in the manner of FIG. 12.

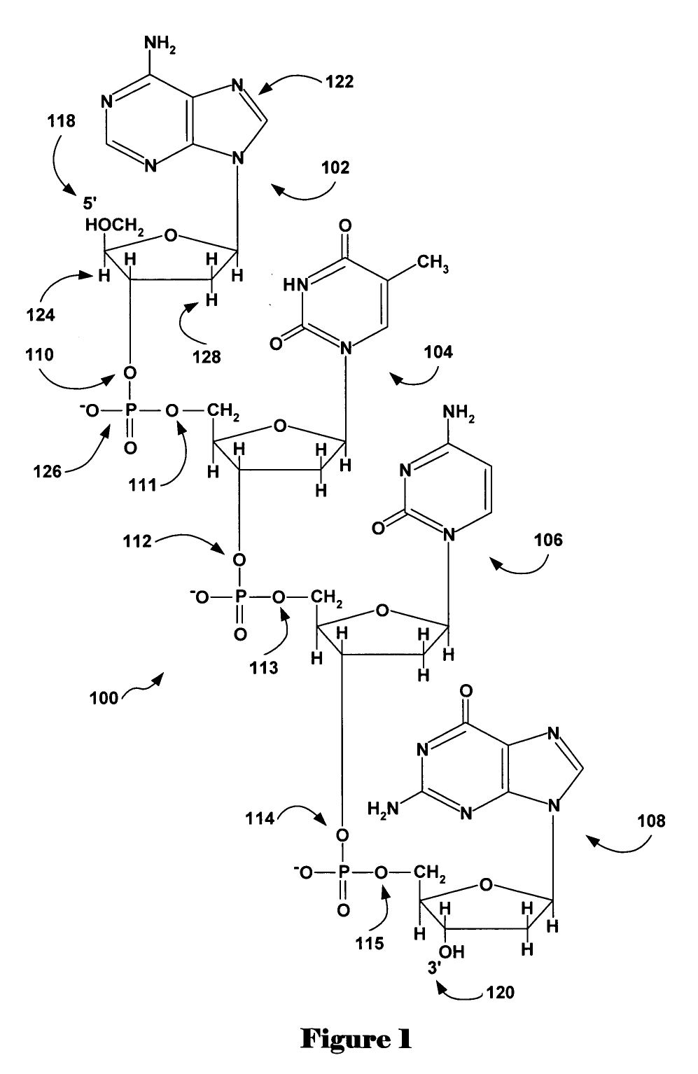

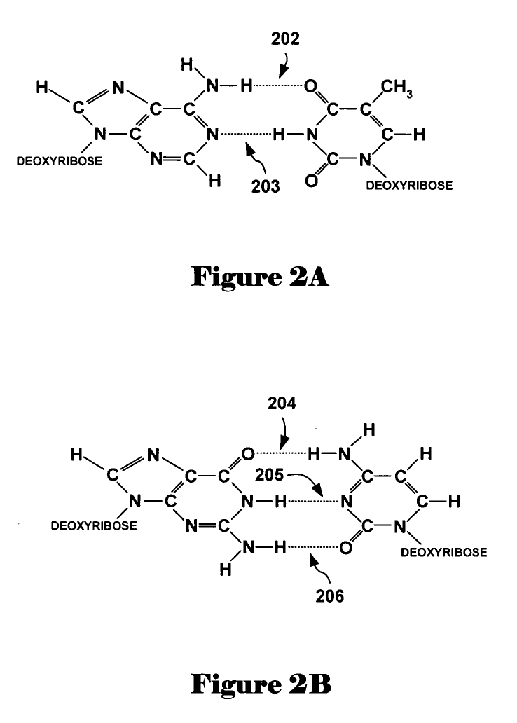

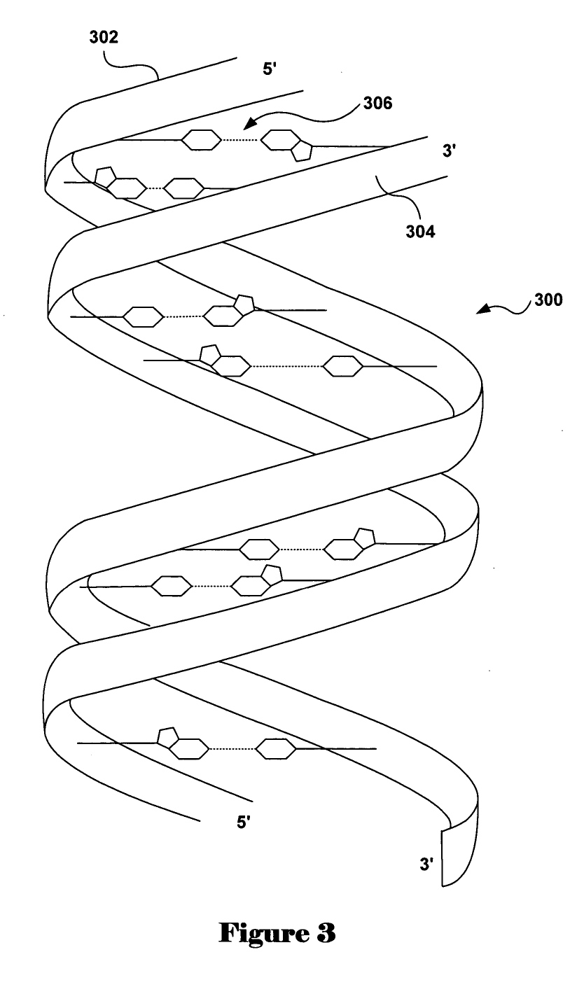

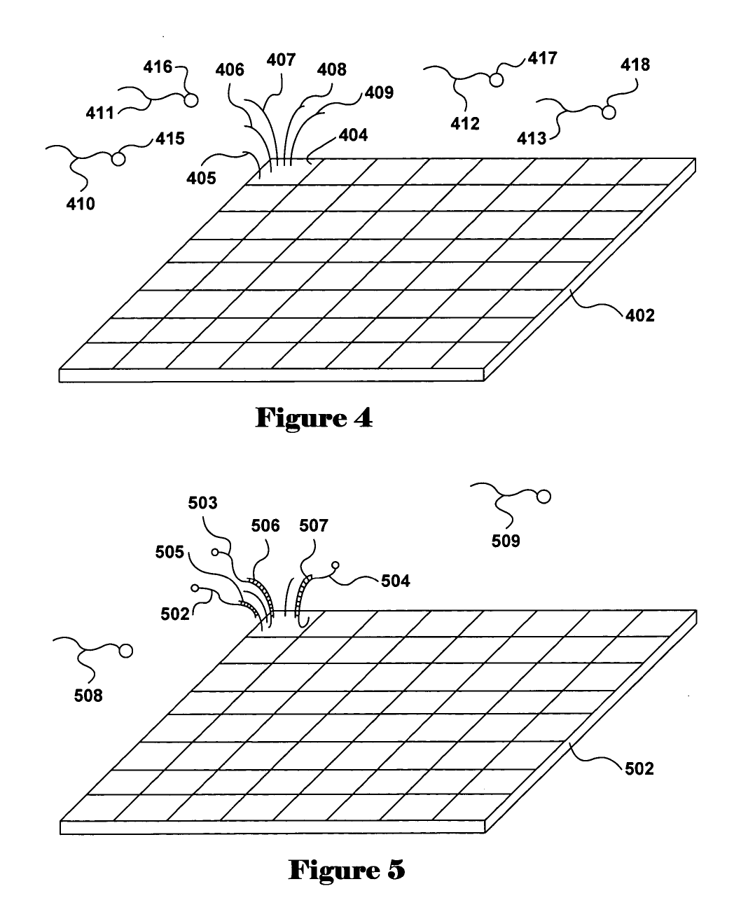

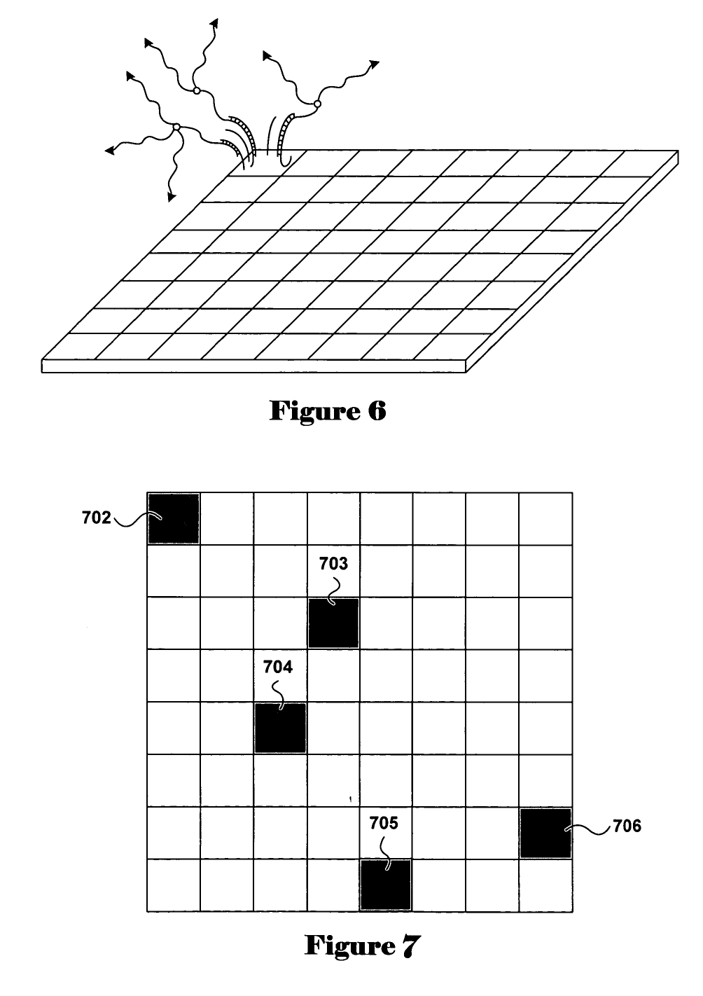

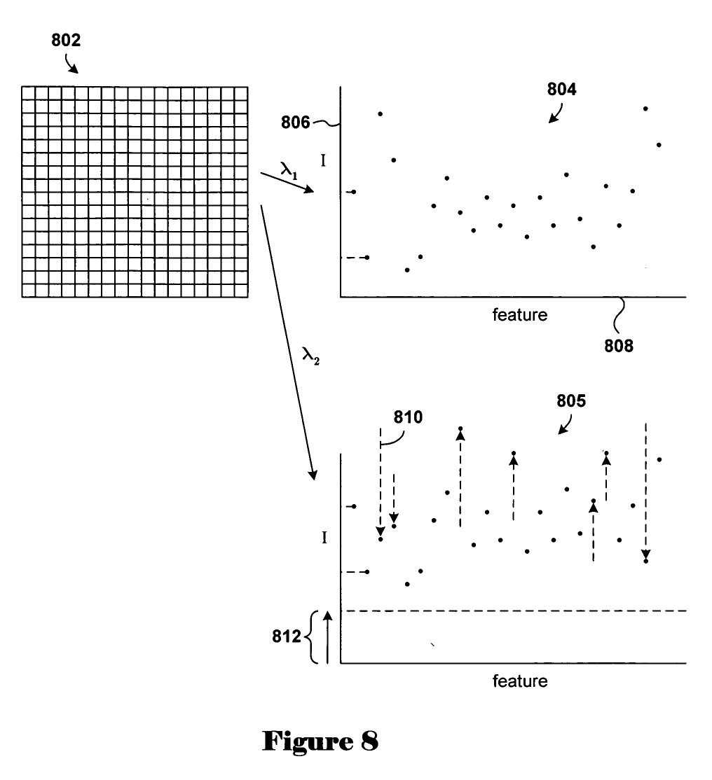

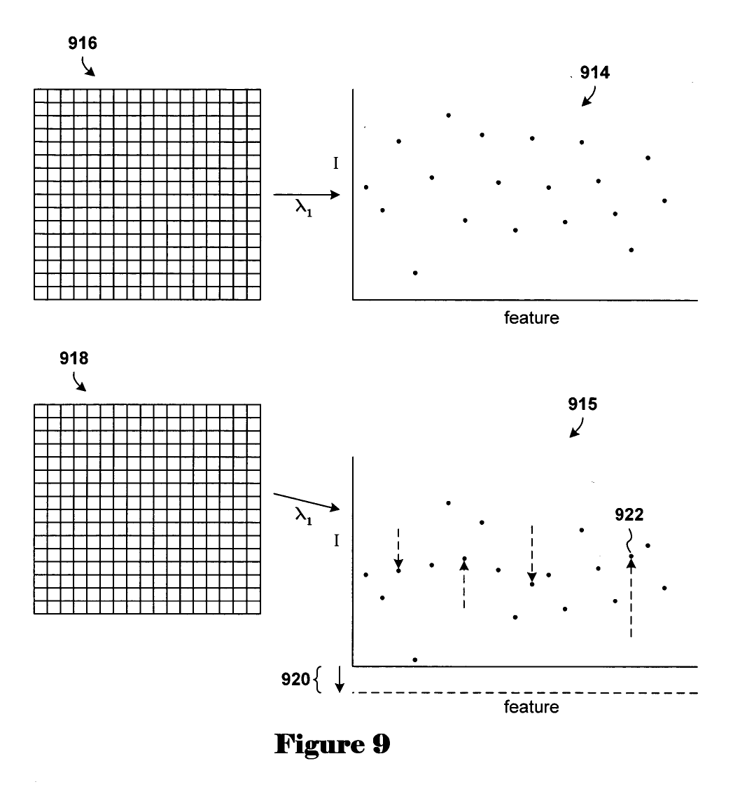

### DETAILED DESCRIPTION OF THE INVENTION

One embodiment of the present invention provides a method and system for normalizing multiple, experimentally related data sets obtain from one or more microarrays. In a first subsection, below, additional information about microarrays is provided. Those readers familiar with microarrays may skip over this first subsection. In a second subsection, the longest-order-preserving-sequence (“LOPS”) technique is described, with reference to graphical representations and a simple example. Finally, in a third subsection, a C++-like pseudocode implementation for a LOPS-based microarray-data normalization system is provided.

### Additional Information About Microarrays

An array may include any one-, two- or three-dimensional arrangement of addressable regions, or features, each bearing a particular chemical moiety or moieties, such as biopolymers, associated with that region. Any given array substrate may carry one, two, or four or more arrays disposed on a front surface of the substrate. Depending upon the use, any or all of the arrays may be the same or different from one another and each may contain multiple spots or features. A typical array may contain more than ten, more than one hundred, more than one thousand, more ten thousand features, or even more than one hundred thousand features, in an area of less than 20 cm² or even less than 10 cm². For example, square features may have widths, or round feature may have diameters, in the range from a 10 μm to 1.0 cm. In other embodiments each feature may have a width or diameter in the range of 1.0 μm to 1.0 mm, usually 5.0 μm to 500 μm, and more usually 10 μm to 200 μm. Features other than round or square may have area ranges equivalent to that of circular features with the foregoing diameter ranges. At least some, or all, of the features may be of different compositions (for example, when any repeats of each feature composition are excluded the remaining features may account for at least 5%, 10%, or 20% of the total number of features). Interfeature areas are typically, but not necessarily, present. Interfeature areas generally do not carry probe molecules. Such interfeature areas typically are present where the arrays are formed by processes involving drop deposition of reagents, but may not be present when, for example, photolithographic array fabrication processes are used. When present, interfeature areas can be of various sizes and configurations.

Each array may cover an area of less than 100 cm², or even less than 50 cm², 10 cm² or 1 cm². In many embodiments, the substrate carrying the one or more arrays will be shaped generally as a rectangular solid having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. Other shapes are possible, as well. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, a substrate may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulsejets of either polynucleotide precursor units (such as monomers) in the case of in situ fabrication, or the previously obtained polynucleotide. Such methods are described in detail in, for example, U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. Other drop deposition methods can be used for fabrication, as previously described herein. Also, instead of drop deposition methods, photolithographic array fabrication methods may be used such as described in U.S. Pat. No. 5,599,695, U.S. Pat. No. 5,753,788, and U.S. Pat. No. 6,329,143. Interfeature areas need not be present particularly when the arrays are made by photolithographic methods as described in those patents.

A microarray is typically exposed to a sample including labeled target molecules, or, as mentioned above, to a sample including unlabeled target molecules followed by exposure to labeled molecules that bind to unlabeled target molecules bound to the array, and the array is then read. Reading of the array may be accomplished by illuminating the array and reading the location and intensity of resulting fluorescence at multiple regions on each feature of the array. For example, a scanner may be used for this purpose, which is similar to the AGILENT MICROARRAY SCANNER manufactured by Agilent Technologies, Palo Alto, Calif. Other suitable apparatus and methods are described in U.S. patent applications: Ser. No. 10/087,447 “Reading Dry Chemical Arrays Through The Substrate” by Corson et al., and Ser. No. 09/846,125 “Reading Multi-Featured Arrays” by Dorsel et al. However, arrays may be read by any other method or apparatus than the foregoing, with other reading methods including other optical techniques, such as detecting chemiluminescent or electroluminescent labels, or electrical techniques, for where each feature is provided with an electrode to detect hybridization at that feature in a manner disclosed in U.S. Pat. No. 6,251,685, U.S. Pat. No. 6,221,583 and elsewhere.

A result obtained from reading an array may be used in that form or may be further processed to generate a result such as that obtained by forming conclusions based on the pattern read from the array, such as whether or not a particular target sequence may have been present in the sample, or whether or not a pattern indicates a particular condition of an organism from which the sample came. A result of the reading, whether further processed or not, may be forwarded, such as by communication, to a remote location if desired, and received there for further use, such as for further processing. When one item is indicated as being remote from another, this is referenced that the two items are at least in different buildings, and may be at least one mile, ten miles, or at least one hundred miles apart. Communicating information references transmitting the data representing that information as electrical signals over a suitable communication channel, for example, over a private or public network. Forwarding an item refers to any means of getting the item from one location to the next, whether by physically transporting that item or, in the case of data, physically transporting a medium carrying the data or communicating the data.

As pointed out above, array-based assays can involve other types of biopolymers, synthetic polymers, and other types of chemical entities. A biopolymer is a polymer of one or more types of repeating units. Biopolymers are typically found in biological systems and particularly include polysaccharides, peptides, and polynucleotides, as well as their analogs such as those compounds composed of, or containing, amino acid analogs or non-amino-acid groups, or nucleotide analogs or non-nucleotide groups. This includes polynucleotides in which the conventional backbone has been replaced with a non-naturally occurring or synthetic backbone, and nucleic acids, or synthetic or naturally occurring nucleic-acid analogs, in which one or more of the conventional bases has been replaced with a natural or synthetic group capable of participating in Watson-Crick-type hydrogen bonding interactions. Polynucleotides include single or multiple-stranded configurations, where one or more of the strands may or may not be completely aligned with another. For example, a biopolymer includes DNA, RNA, oligonucleotides, and PNA and other polynucleotides as described in U.S. Pat. No. 5,948,902 and references cited therein, regardless of the source. An oligonucleotide is a nucleotide multimer of about 10 to 100 nucleotides in length, while a polynucleotide includes a nucleotide multimer having any number of nucleotides.

As an example of a non-nucleic-acid-based microarray, protein antibodies may be attached to features of the array that would bind to soluble labeled antigens in a sample solution. Many other types of chemical assays may be facilitated by array technologies. For example, polysaccharides, glycoproteins, synthetic copolymers, including block copolymers, biopolymer-like polymers with synthetic or derivitized monomers or monomer linkages, and many other types of chemical or biochemical entities may serve as probe and target molecules for array-based analysis. A fundamental principle upon which arrays are based is that of specific recognition, by probe molecules affixed to the array, of target molecules, whether by sequence-mediated binding affinities, binding affinities based on conformational or topological properties of probe and target molecules, or binding affinities based on spatial distribution of electrical charge on the surfaces of target and probe molecules.

Scanning of a microarray by an optical scanning device or radiometric scanning device generally produces a scanned image comprising a rectilinear grid of pixels, with each pixel having a corresponding signal intensity. These signal intensities are processed by an array-data-processing program that analyzes data scanned from an array to produce experimental or diagnostic results which are stored in a computer-readable medium, transferred to an intercommunicating entity via electronic signals, printed in a human-readable format, or otherwise made available for further use. Microarray experiments can indicate precise gene-expression responses of organisms to drugs, other chemical and biological substances, environmental factors, and other effects. Microarray experiments can also be used to diagnose disease, for gene sequencing, and for analytical chemistry. Processing of microarray data can produce detailed chemical and biological analyses, disease diagnoses, and other information that can be stored in a computer-readable medium, transferred to an intercommunicating entity via electronic signals, printed in a human-readable format, or otherwise made available for further use.

### n-Dimensional Lops Normalization Technique

Data points, or features, in a number of microarray data sets have both identities and values. The values of a data point are generally a measure of scanned intensities of light or radiation emitted from labeled target molecules bound to the feature, and the identity may be two-coordinate indexes, a sequence number, or an alphanumeric label that uniquely identifies the feature within the data set. A data point may also, in certain cases, be associated with a weight, where the weight expresses a measure of confidence, constancy, or some other parameter or characteristic.

An order-preserving sequence is a sequence of data points in which the values of the data points within the sequence uniformly increase within the sequence. When a sequence is defined as an ordered subset of points within a data set, then a longest-order-preserving sequence (“LOPS”) is the maximally sized, one or more ordered subsets of points selected from the data set that are ordered by signal strength or by some other associated value, parameter, or characteristic. A heaviest-order-preserving sequence (“HOPS”) is the order-preserving sequence with greatest sums of weights associated with data points in order-preserving sequence. The method that represents one embodiment of the present invention, discussed below, is useful for calculating multi-data-set LOPS, and may be simply extended for calculating multi-data-set HOPS. The method is discussed with respect to LOPS, but the present invention comprehends both calculation of LOPS and HOPS.

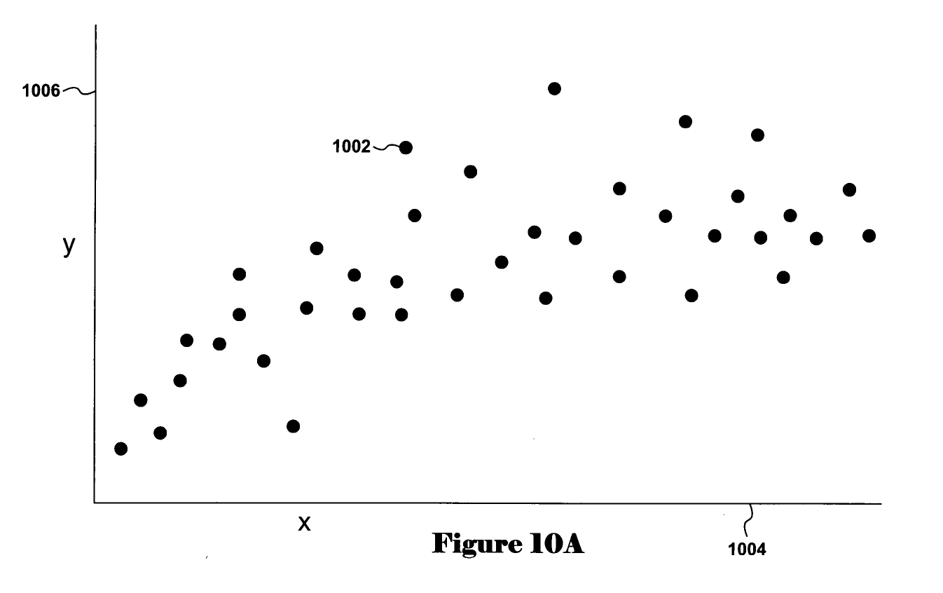

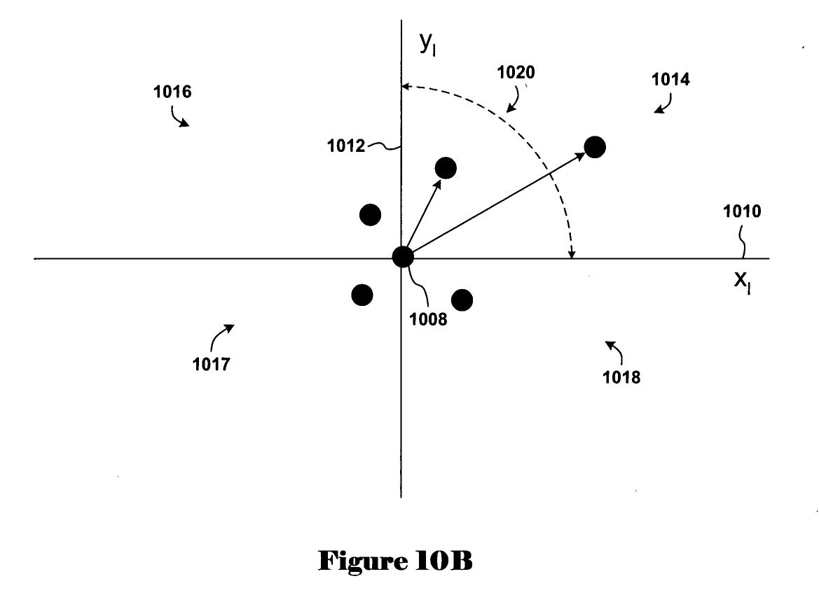

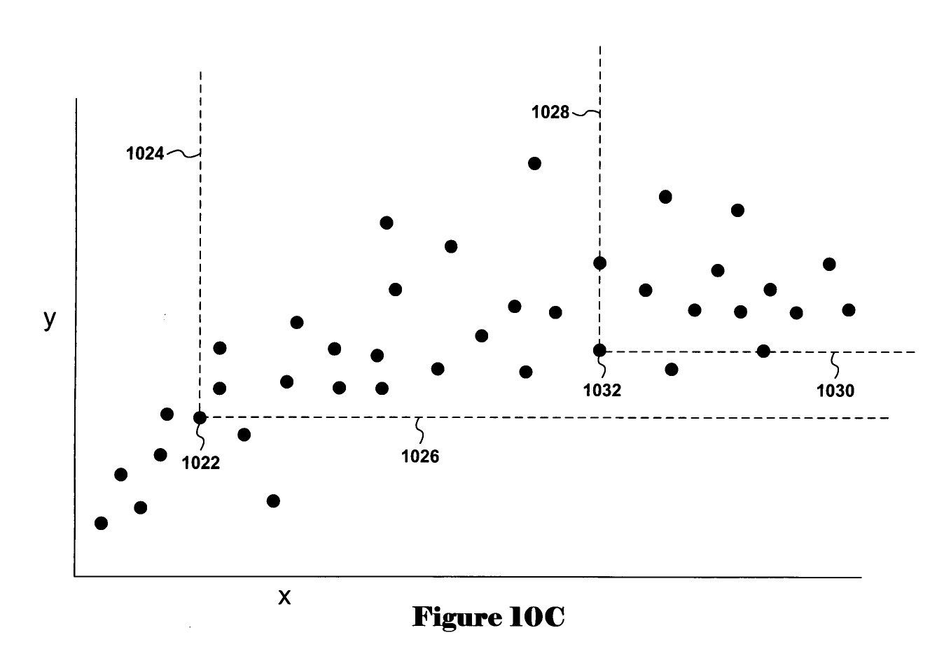

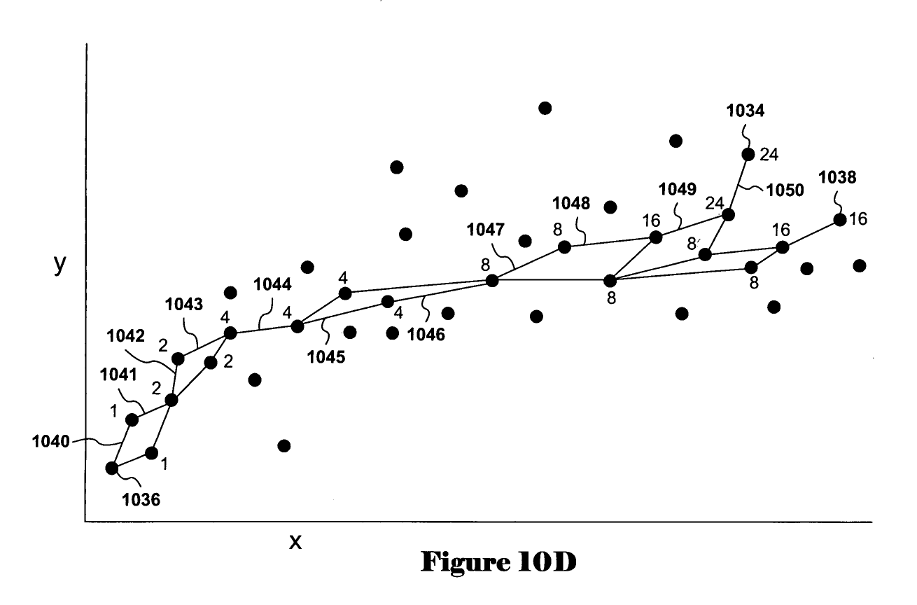

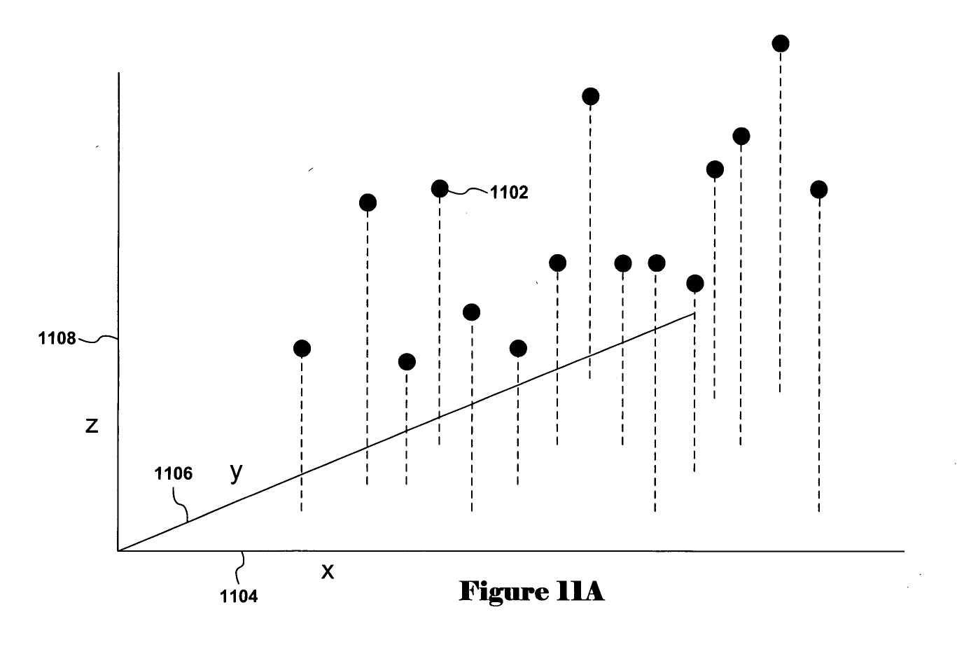

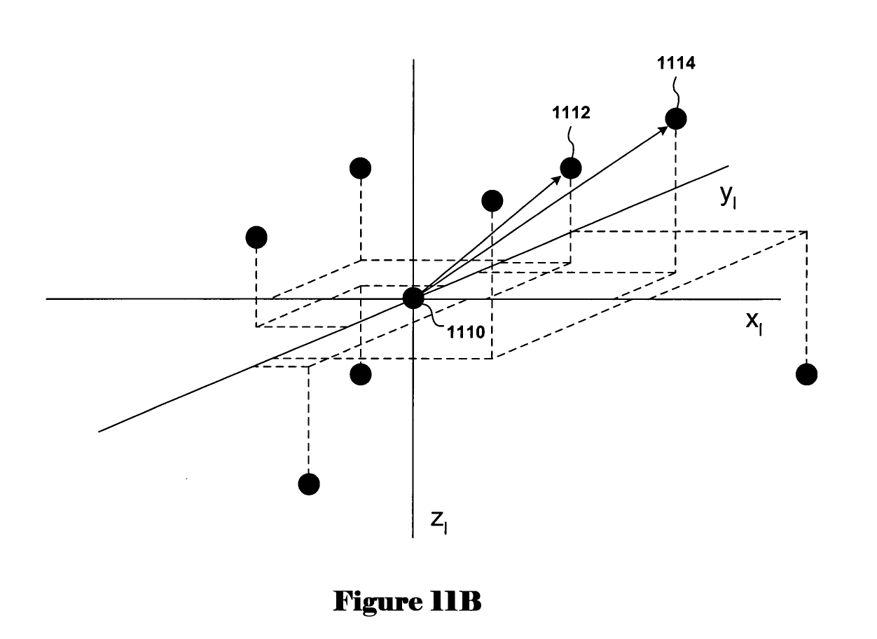

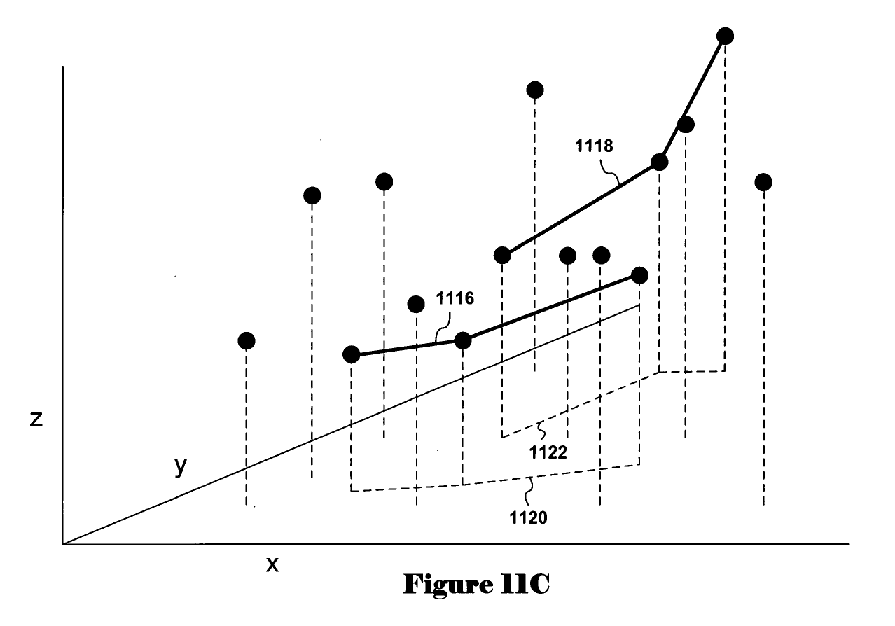

FIGS. 10A-D illustrate the two-dimensional LOPS. In FIG. 10A, a number of data points, such as data point 1002, are distributed in a two-dimensional space defined by an orthogonal coordinate system. The positive, horizontal axis 1004 corresponds to a first coordinate x, and the vertical axis 1006 in FIG. 10A is the positive axis for the coordinate y. Each data point, such as data point 1002, has an identity as well as an x value and a y value represented by the position of the data point within the two-dimensional space. Commonly, the data points are associated with Cartesian coordinates (x, y) where x is the value of the data point with respect to the x axis, and y is the value of the data point with respect to the y axis. Such a two-dimensional distribution may arise from scanning a microarray at two different frequencies, with the x values representing signal intensities scanned at one frequency, and the y values representing signal intensities scanned at another frequency.

In order to determine a LOPS for a two-dimensional distribution of data points, such as that shown in FIG. 10A, a means of establishing the order of data points must be first defined. FIG. 10B illustrates one technique for establishing the order of data points within a two-dimensional distribution, such as that shown in FIG. 10A. In FIG. 10B, a data point 1008 is shown at the position (0,0) with respect to local, orthogonal axes x₁ 1010 and y₁ 1012. Any data point in the first quadrant of the two-dimensional space defined by orthogonal axes x₁ and y₁ is considered to be greater than, or having a higher order than, data point 1008. Data points within the diagonally opposite quadrant 1017 of the two-dimensional space are considered to be less than, or of smaller order than, data point 1008. Data points in the neighboring quadrants 1016 and 1018 are not considered, as they are not directly comparable by a less-than/greater-than ordering, so the defined ordering is a partial ordering. In other words, an order-preserving sequence, or path, in a two-dimensional space defined by orthogonal coordinates has path edges that point in a 90-degree 1020 range of directions between east, corresponding to the direction of the x-coordinate axis, and north, corresponding to the direction of the positive y-coordinate axis.

As shown in FIG. 10C, the definition of order illustrated in FIG. 10B is applied to each data point within the data point distribution and selects a subset of data points that are greater than any particular data point. For example, all data points to the right of local y axis 1024 and above local x axis 1026 are considered to be greater than data point 1022, and all data points to the right of local y axis 1028 and above local x axis 1030 are considered to be greater than data point 1032. This definition allows for constructing order-preserving sequences within a two-dimensional distribution of data points. Since the signals in each dimension are compared within each dimension independently, only the ranking of the points is necessary to determine the LOPS and HOPS sequences. Thus, the ranks of the points in each dimension can be substituted for their real experimental values. This is useful for implementations on possessor and/or operating systems on which integer calculations can be performed more efficiently than floating point.

FIG. 10D illustrates construction of a number of longest order-preserving sequences within the distribution of data points shown in FIG. 10A. It turns out that, in general, there may be many different longest order-preserving sequences that all have the same, maximal length, within a two-dimensional distribution of data points. In other words, unlike in the one-dimensional case, there may be many different LOPS within a two-dimensional data-point distribution. In FIG. 10B, the numerical label associated with certain data points, such as numerical label “24” associated with data point 1034, indicates the number of different longest order-preserving sequences or subsequences that begin with data point 1036 and end with the data point having the associated numerical label. Thus, there are 24 longest order-preserving sequences of length 12 (where length equals the number of nodes in a sequence) that begin with data point 1036 and end with data point 1034. There are 16 different LOPS that begin with data point 1036 and end with data point 1038. 40 different LOPS with maximal length 12 are shown within the two-dimensional data distribution of FIG. 10D. Any one of the 40 different LOPS in FIG. 10D can be constructed from edges 1040-1050. It should be noted that, although in the example shown in FIG. 10D, all LOPS begin with the single data point 1036, all LOPS constructed from a general data-point distribution need not begin with a particular point.

FIGS. 11A-C illustrate a LOPS within a three-dimensional distribution of data points. FIG. 11A shows a number of data points, such as data point 1102, distributed within the positive octant of a three-dimensional space defined by the three mutually orthogonal axes x 1104, y 1106, and z 1108. In a three-dimensional distribution, each data point, such as data point 1102, has an identity as well as values with respect to the x, y, and z axes. In order to construct a LOPS within a three-dimensional data-point distribution, the definition of a positive direction, or order, for data points is needed, as it was needed in the two-dimensional and one-dimensional cases. FIG. 11B illustrates a positive direction for construction of a LOPS in a three-dimensional distribution. In FIG. 11B, a data point 1110 is shown positioned at the origin of three, mutually orthogonal, local axes x₁, y₁, and z₁. Any data points within the positive octant bounded by the positive local axes x₁, y₁, and z₁, is considered to be greater than, or of higher order than, data point 1110, while data points within the negative octant bounded by the negative local axes \overline{x}₁, \overline{y}₁, \overline{z}₁, is considered to be less than, or of lower order than, data point 1110, and data points in all six other octants are considered neither less than nor greater than data point 1110. Thus, in FIG. 11B, data points 1112 and 1114, located within the positive octant, are considered to be greater than data point 1110, and would be candidates for constructing the next edge of an order-preserving sequence passing through data point 1110.

FIG. 11C shows two LOPS constructed from the three-dimensional distribution of data points shown in FIG. 11A. Projections of the two LOPS 1116 and 1118 onto the xy plane 1120 and 1122, respectively, are also shown in FIG. 11C. Interestingly, as the number of dimensions increases, the ratio of the length of one or more LOPS within a generalized distribution of data points to the number of data points within the distribution dramatically decreases. In a one-dimensional case, all the data points are members of a single LOPS within a data-point distribution. In the exemplary case of a two-dimensional distribution, a relatively large fraction of the data points of the distribution are included in one or more of the 40 different LOPS, but the length of a LOPS in a two-dimensional case is significantly less than the total number of data points within a two-dimensional distribution. In the three-dimensional case, illustrated in FIGS. 11A-C, the length of each LOPS is only three nodes, considerably less than the total number of data points shown in the distribution. Thus, as the number of dimensions increase, the constraints implied by a longest-order-preserving sequence become increasingly severe, and result in selecting a smaller and smaller percentage of the total number of data points within a distribution. Of course, in certain special distributions, the points may be arranged in any n-dimensional case so that all points occur within the LOPS, as in the one-dimensional case. However, for generalized distributions, the severity of the constraints increases with increasing dimensions. This is mathematically reasonable because, as can be seen by comparing FIG. 10B to FIG. 11B, the number of parameters necessary to determine the relative directions of one data point with respect to another increases linearly with an increase in dimensions. As the number of dimensions increases, and as the noise associated with each dimension increases, there are fewer and fewer points in each LOPS sequence, eventually leading to high-dimension cases in which the only LOPS sequences are trivial, single-point sequences. Such extrema represent over-constrained systems.

An approach to reducing the problem of over-constrained systems with increasingly higher dimensionality is the use of LOPS Scoring. The LOPS within a distribution can be thought of as being roughly analogous to a kind of centroid of the distribution. The order-preserving constraint implies that the LOPS is constrained to approximately correspond to the shape of a distribution of data points, and the longest constraint forces the LOPS to coincide with the most densely populated portions of a distribution. Assuming that the majority of data points within two or more different data sets are not systematically perturbed in a series of experiments, analogous to assuming a majority of genes are housekeeping genes within a total number of genes monitored in a gene-expression experiment, then those data points coincident with, or lying near to, a LOPS within a distribution of data points in n-dimensions, where n is the number of data sets, is reasonably expected to select those data points most likely to be unperturbed, or relatively constant, over the total number of data sets.

One embodiment of the present invention is selection of data points within an n-dimensional distribution of data points for normalization that coincide with, or fall closely to, a LOPS, or the set of data points coincident with one or more LOPS, within an n-dimensional distribution. However, to be practical, there must be a relatively efficient computational method for calculating sets of LOPS points from n different data sets. Moreover, because of the increasing constraint represented by selecting points coincident with one or more LOPS as the number of dimensions increases, the computational method needs to be able to systematically relax, to some degree, the LOPS constraints in order to acquire a sufficient number of normalization data points for statistical reliability of the subsequent normalization process.

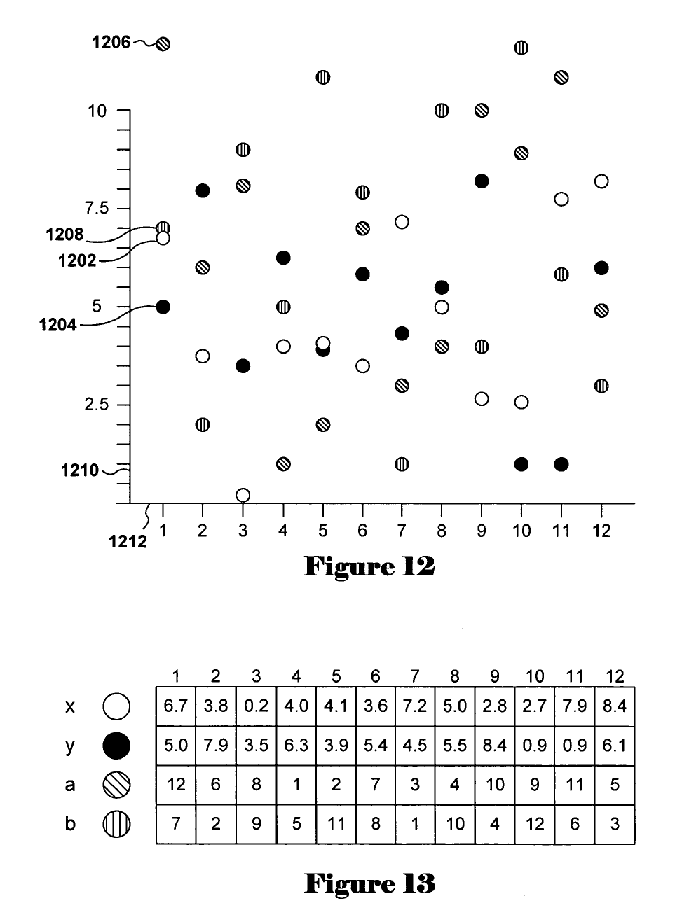

An efficient computational method that represents a portion of one embodiment of the present invention is now described. FIG. 12 shows a distribution of the data points within four data sets x, y, a, and b representing four different samples or channels in a microarray study for a simple example used to illustrate the computational technique that constitutes a portion of one embodiment of the present invention. In FIG. 12, the data points of each data set are indicated by different markings. The data points of the first data set x are represented by unfilled circles or disks, such as unfilled circle 1202. The data points of the second data y set are represented by filled circles or disks, such as filled circle 1204. The data points of the third data set a are represented by crosshatched disks, with the crosshatches sloping diagonally to the right, such as crosshatched disk 1206. The data points of the fourth data set b are represented by vertically crosshatched disks, such as vertically crosshatched disk 1208. The vertical axis 1210 corresponds to the signal intensity measured for a feature of a microarray, and thus represents the value of the data point. The horizontal axis 1212 represents the identity of the feature. Thus data points 1202, 1204, 1206, and 1208 represent the signal intensities measured for feature 1 in the four different data sets. In essence, the four data sets x, y, a, and b are plotted two-dimensionally in FIG. 12, representing a four-dimensional distribution of four-dimensional data points, with the intensity measured for each data set representing the value for a four-dimensional data point in each dimension and the feature corresponding to the plotted data points corresponding to the identity of the four-dimensional data point. It is the goal of the computational method to efficiently, in terms of computation steps or time, select a four-dimensional LOPS within this four-dimensional distribution of data points. The numeric values of the data points in each dimension x, y, a, and b, for each feature, are shown in FIG. 13. Note that, for ease of calculation, two-digit floating point data values are used for the x and y dimensions, while the a and b dimensions have simple one- and two-digit integer values. The method is quite general, and, with properly defined comparison functions, can be used with dimensions each having values selected from quite different data types.

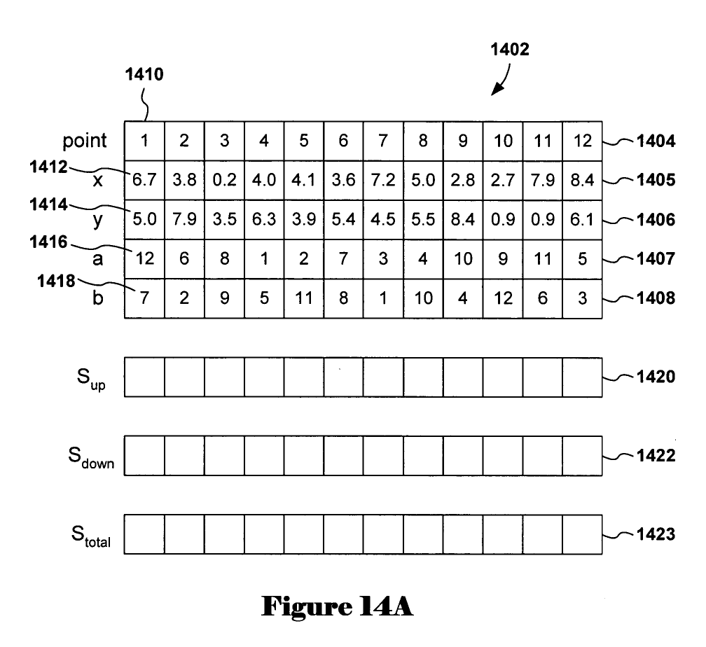

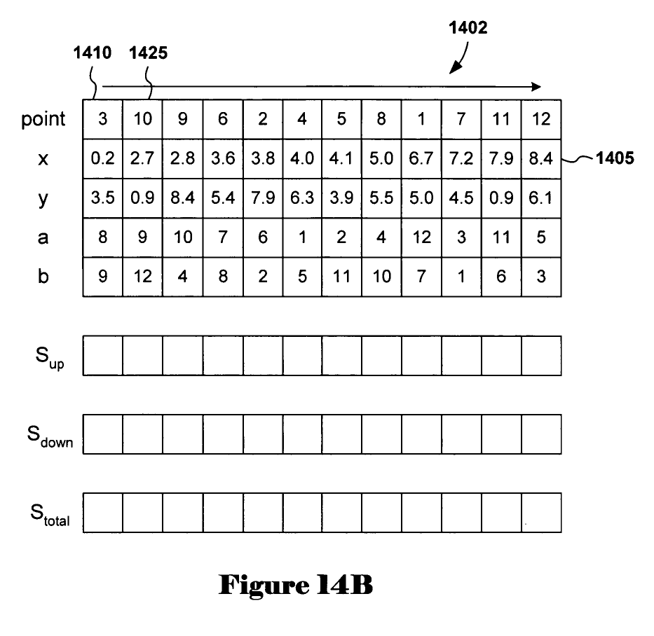

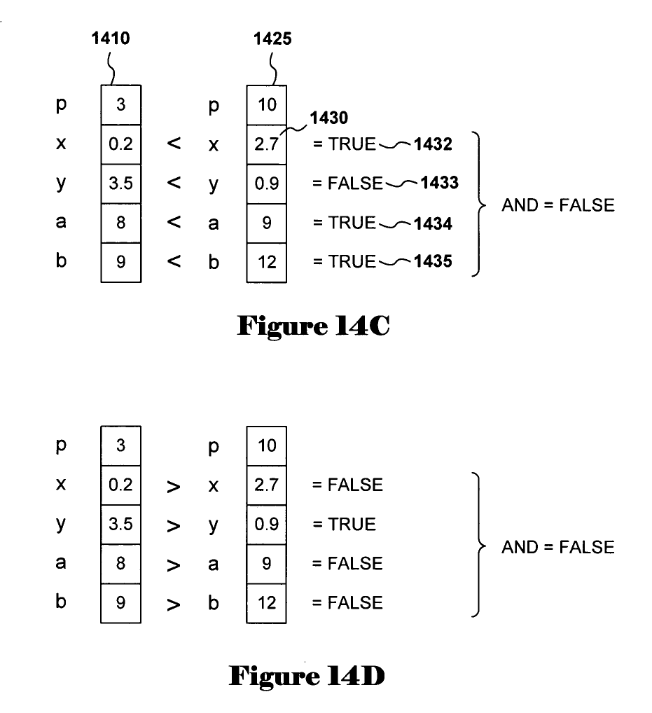

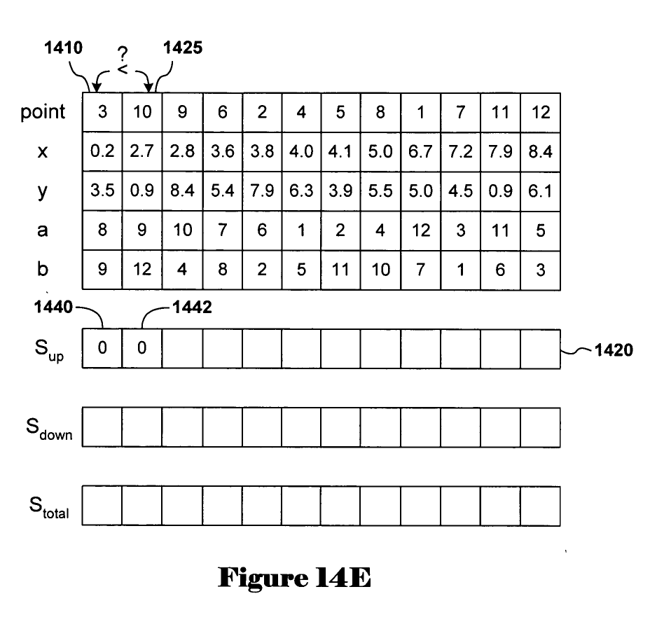

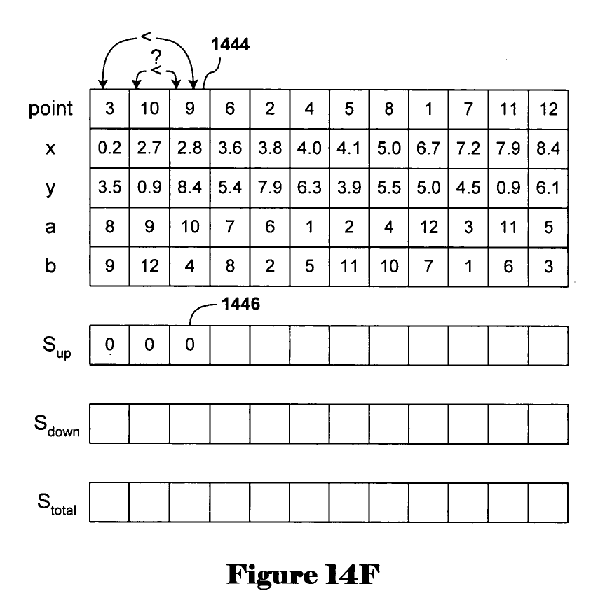

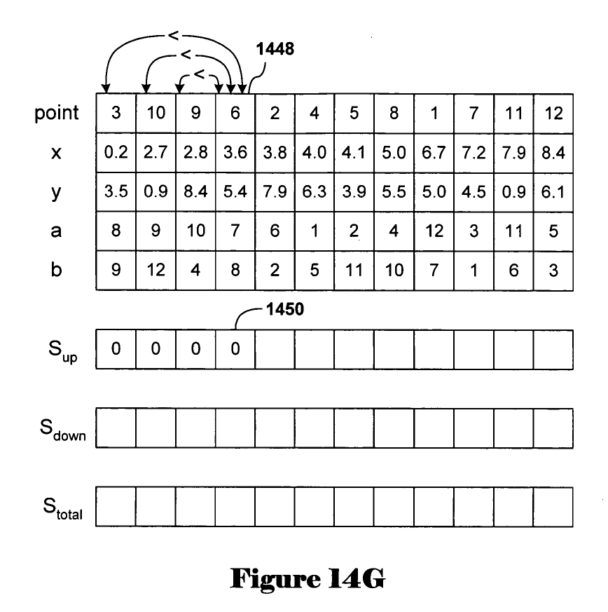

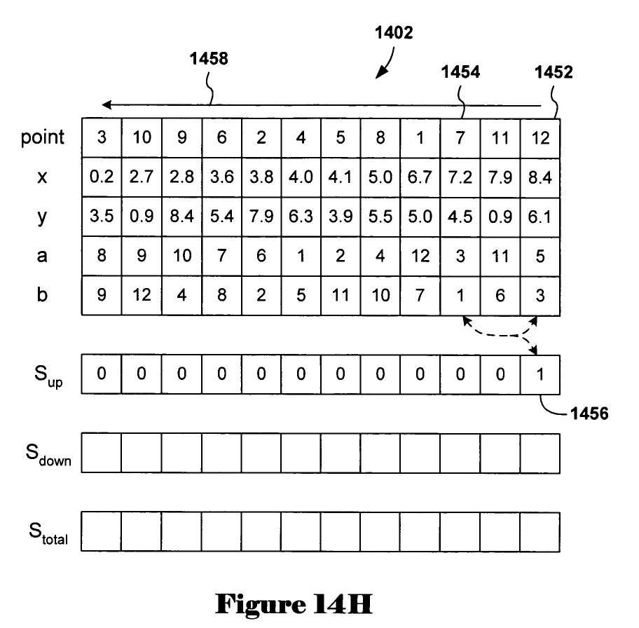

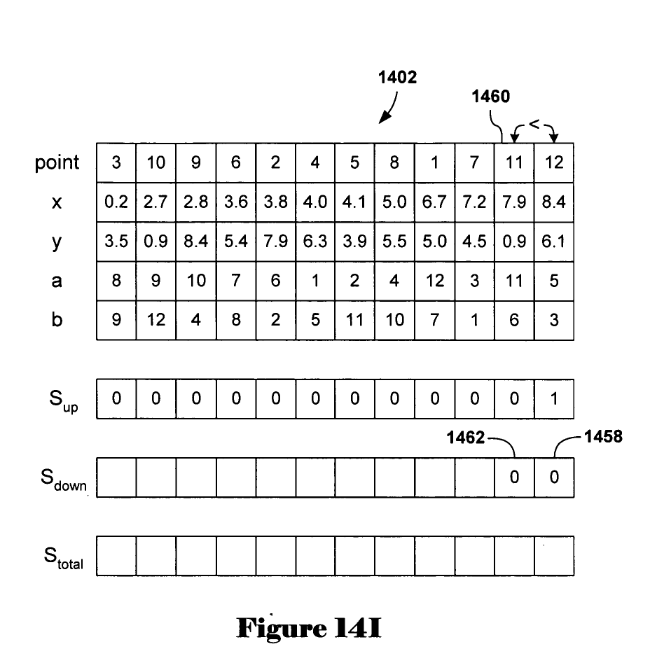

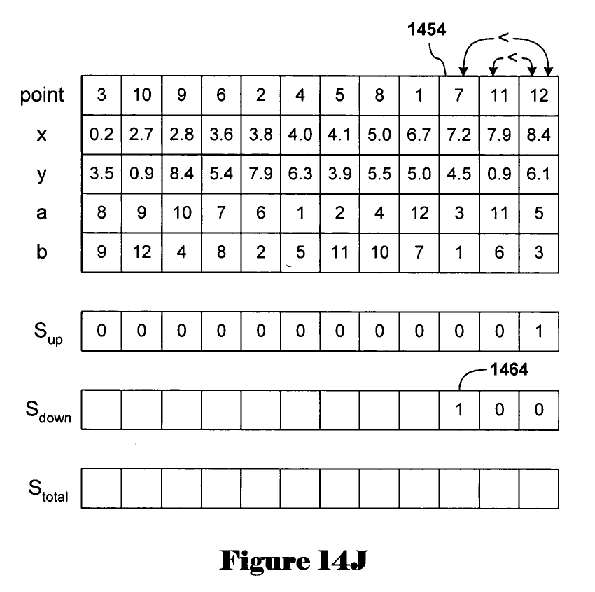

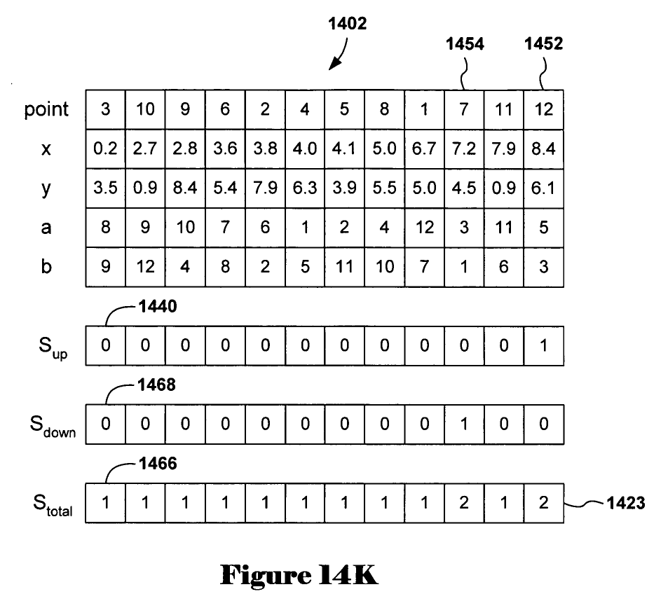

FIGS. 14A-K illustrate computation of a set of points coincident with longest-order-preserving sequences within the data-point distribution shown in FIG. 12 and plotted in FIG. 13. Note that FIGS. 14A-K illustrate finding LOPS, but a similar computational method may be employed to find a HOPS. In fact, LOPS computation is a subset of HOPS computation, with the weight for each data point in LOPS computation assigned the value “1. ” The majority of FIGS. 14A-K employ the illustrative conventions employed in FIG. 14A. The data points, including identities and values, are contained within a first five-row matrix 1402. The identities of the data points are contained within the first row 1404 of the matrix, the values with respect to each of the four dimensions, or four data sets, are stored in subsequent rows 1405-1408. Comparing the first column 1410 of the five-row matrix in FIG. 14A with the data-point distribution plotted in FIG. 12, it can be seen that the first column 1410 corresponds to the data points plotted for feature 1 in FIG. 12, with data point 1202 corresponding to the value “6.7” 1412, data point 1204 corresponding to the value “5.0” 1414, data point 1206 corresponding to the value “12” 1416, and data point 1208 corresponding to the value “7” 1418. The columns of the five-row matrix may be reordered during execution of the computational method. Three one-dimensional arrays 1420, 1422, and 1423, are also shown in FIG. 14A, labeled “Sᵤₚ,” “S_{down},” and “Sₜₒₜₐₗ,” respectively. The purposes of these three one-dimensional arrays are described, below, with respect to subsequent figures.

In FIG. 14B, the method begins by reordering or sorting the columns of the five-row matrix 1402 with respect to the values associated with the data set x, or the x dimension. Note in FIG. 14B that, in the reordered five-row matrix, the values in the row 1405 containing the x-data-set values increase in right-to-left order. The method next involves considering each data point, or column, in the five-row matrix 1402, starting with the second column 1425, and comparing the considered column with columns to the left of the currently considered column.

The comparison used in the method is illustrated in FIGS. 14C-D. For example, FIG. 14C shows the comparison of column 1425 with column 1410, the first step in the rightward traversal of the columns of the five-row matrix 1402. As shown in FIG. 14C, each value within column 1425, such as value “2.7” 1430, is compared with the corresponding value in column 1410. In this case, the comparison is a less than comparison, denoted by the mathematical symbol “<.” The result of the comparison is either the Boolean value TRUE or the Boolean value FALSE. Thus, four pair-wise comparisons of the values in column 1425 with the corresponding values in column 1410 produce the four Boolean values TRUE 1432, FALSE 1433, TRUE 1434, and TRUE 1435. The Boolean values are then combined using the Boolean AND operation to produce the final Boolean value FALSE. In other words, for the data point represented by column 1410 to be less than the data point represented by column 1425, each value associated with the data point represented by column 1425 must be greater than the corresponding value associated with the data point represented by column 1410. This proves to be a rather severe constraint, and corresponds to an extension, to four dimensions, of the ordering constraints illustrated in FIGS. 10B and 11B for the two-dimensional and three-dimensional data point distributions discussed above. FIG. 14D shows the similar greater than comparison operation designated by the comparison symbol “>.”

As shown in FIG. 14E, a zero is placed into the first element 1440 of the Sᵤₚ array 1420. Next, the currently considered column, in this case the initially currently considered column 1425, is compared to each column to the left using the comparison operation illustrated in FIG. 14C. If the four-dimensional data points represented by any column to the left of the currently considered column, or data point, are less than the currently considered data point, then the entry in the Sᵤₚ array 1420 corresponding to the currently considered column (element 1422 for column 1425, for example) is set to the integer value one greater than the maximum value in the Sᵤₚ array corresponding to all data points to the left of, and less than, the currently considered data point. As illustrated in FIG. 14D, the comparison of data points 10 and 3, represented by columns 1425 and 1410, respectively, leads to the Boolean value FALSE. Thus there are no data points to the left of the currently considered data point that are less than the currently considered data point, and so the value “0” is placed into element 1442 of the Sᵤₚ array 1420 corresponding to column 1425.

In the next step of the method, the currently considered data point is shifted, one data point to the right, to the data point represented by column 1444. This data point is separately compared to each data point to its left to determine whether any of the data points to the left are less than the currently considered data point. None are, so the value “0” is placed into the Sᵤₚ element 1446 corresponding to column 1444.

Again, as shown in FIG. 14G, the currently considered data point is shifted one to the right, and the currently considered data point is then compared to each data point to its left to determine whether any of the data points to its left are less than the currently considered data point. The three pair-wise comparisons, employing the less than comparison operation described with reference to FIG. 14C, reveal that none of the data points to the left of the currently considered data point, represented by column 1448, is less than the data point represented by column 1448. Therefore, yet another zero is placed into the Sᵤₚ array in element 1450 corresponding to data point “6” represented by column 1448.

This process continues until all the entries in the Sᵤₚ array are filled, as shown in FIG. 14H. Note that of all the pair-wise comparisons of data points in this first, rightward traversal of the five-row matrix 1402, only one comparison, that between data points 12 and 7 represented by columns 1452 and 1454, respectively, results in a left-lying data point being considered less than a data point to its right. Thus, only a single value greater than zero, namely the value “1,” occurs in the completed Sᵤₚ array in element 1456 corresponding to data point “12,” represented by column 1452. Then, as indicated by the arrow 1458 in FIG. 14H, a leftward traversal of the five-row matrix 1402 is carried out. In this traversal, each data point, starting with the data point represented by the second-most right-hand data point in the five-row matrix, is compared with all data points to its right in the five-row matrix. When the currently considered data point is less than any of the data points to its right, then a value one greater than the maximum value of any data points to the right of the currently considered data point, and greater than the currently considered data point, in the S_{down} array is entered into the S_{down} array element corresponding to the currently considered data point. The rightmost element of the S_{down} array 1458 is set to the value zero prior to the traversal. As shown in FIG. 14I, the first comparison of initially considered data point “11,” represented by column 1460 in matrix 1402, with the data point to its right reveals that data point “11” is not less than data point “12,” and thus the value “0” is placed into the corresponding element 1462 of the S_{down} array.

Next as shown in FIG. 14J, the currently considered data point is shifted to the left by one column to column 1454, and pair-wise compared to each data point represented by columns to the right of the currently considered data point. In this case, currently considered data point “7,” represented by column 1454, is less than data point “12, ” and so the value “1,” an integer value one greater than any value to the right of the element of the S_{down} array corresponding to the currently considered data point, is placed into element 1464 of the S_{down} array corresponding to currently considered data point “7.”

As shown in FIG. 14K, the leftward traversal of the five-row matrix 1402 proceeds until all elements of the S_{down} array have been assigned values. In a final step in searching for a LOPS, the values of the Sₜₒₜₐₗ array are assigned to be one greater than the sum of the values in corresponding elements of the Sᵤₚ and S_{down} arrays. Thus, for example, the value “1” in element 1466 of the Sₜₒₜₐₗ array 1423 represents one greater than the sum of the values stored in elements 1440 and 1468 of the Sᵤₚ and S_{down} arrays, respectively. The values in the Sₜₒₜₐₗ array are the lengths of the longest-order-preserving sequences to which each of the corresponding data points can be assigned. Thus, as determined by the method illustrated in FIGS. 14A-K, the LOPS for the four-dimensional data distribution corresponding to the plot of four data sets shown in FIG. 12 has a length of two, and includes data points “7” and “12” represented by columns 1452 and 1454 of the five-row matrix 1402. When there is only one LOPS, the LOPS set can be selected from the data points by determining the maximum value in the Sₜₒₜₐₗ array and selecting data points with entries in the Sₜₒₜₐₗ array equal to the maximum value. Points lying close to the LOPS may be selected by selecting those points with values in the Sₜₒₜₐₗ array nearly equal to the maximum value. When there are more than one LOPS, any particular LOPS sequence can be selected by incorporating pointers into the Sᵤₚ and S_{down} arrays, and following pointer chains. However, there is not necessarily a need to choose a particular LOPS. One can select data points with values in the Sₜₒₜₐₗ array greater than a threshold value, for example, or randomly select a particular number of, or fraction of, the data points with values in the Sₜₒₜₐₗ array greater than a threshold value. A data point in any LOPS is a reasonable candidate for a normalizing data point.

The constraints implied by selecting a true LOPS set from the four-dimensional data-point distribution are too great. It would be desirable to select a larger set of points for normalization purposes. Although the current example includes very few data points, and the severity of the constraint represented by selecting LOPS sets is not readily apparent, a general four-dimensional distribution containing hundreds of points might be expected to lead to a LOPS set containing only a very few data points.

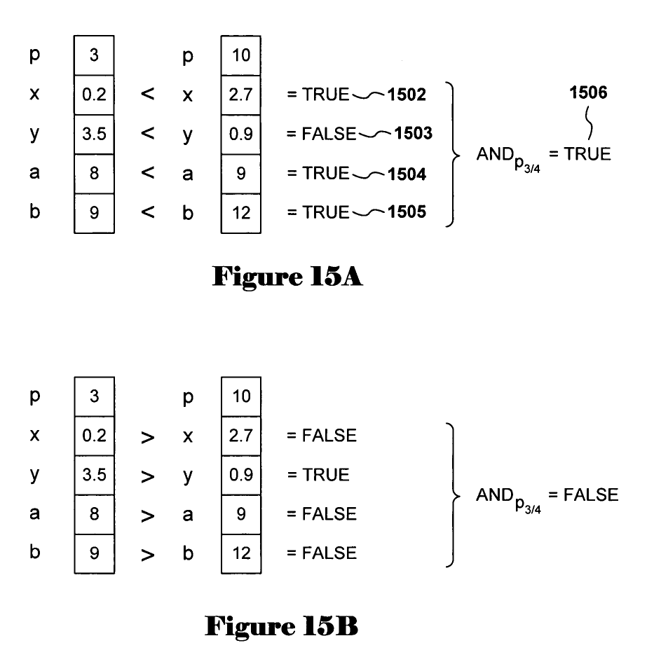

In order to relax the constraints implicit in selecting the LOPS set, as illustrated in FIG. 14A-K, a different comparison operation may be employed. FIGS. 15A-B illustrated a relaxed comparison in which only three of the four values of a first data point must exceed the corresponding values of a second data point in order for the first data point to be considered to be greater than the second data point. FIG. 15A shows the less than comparison of data point 3 to data point 10 using the relaxed comparison operation. In this case, the pair-wise comparisons of corresponding values produce the Boolean values TRUE 1502, FALSE 1503, TRUE 1504, and TRUE 1505. In the relaxed comparison, only three of the four Boolean values need to be TRUE. Therefore, unlike in the strict comparison operation illustrated in FIG. 14C, the final result produced by the relaxed comparison is TRUE 1506. An analogous greater than, relaxed comparison is illustrated in FIG. 15B. Note that the comparison operations shown in FIGS. 15A-B relax one of four dimensions. An even more relaxed comparison operation may relax two of four dimensions. In an n-dimensional comparison, 1, 2, . . . n−1 different comparison operators may be generated by relaxing 1, 2, . . . n−1 dimensions. Even more complex comparison operations can be imagined. For example, if one data set is known to be less reliable than the other data sets in a multi-data-set normalization, then the less-reliable dimension may be relaxed, or not considered. Statistically based relaxations may also be employed.

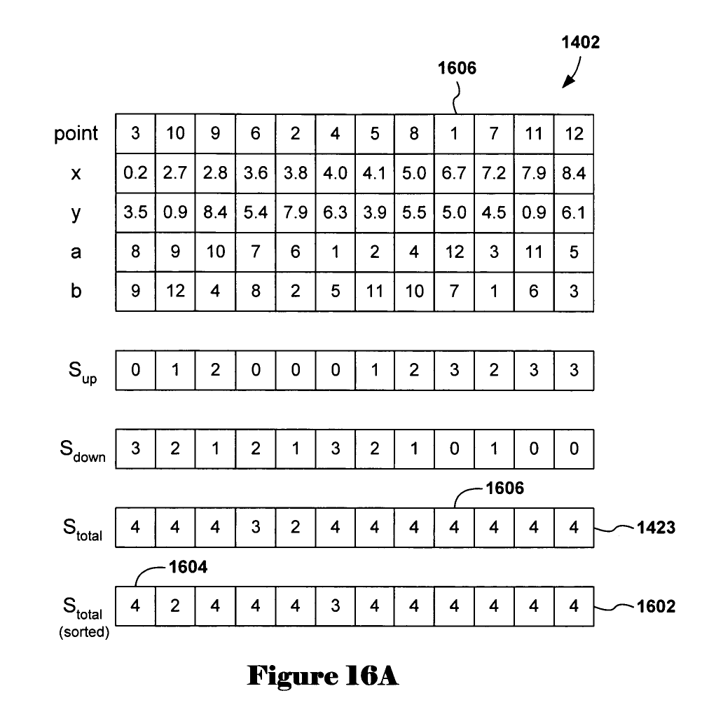

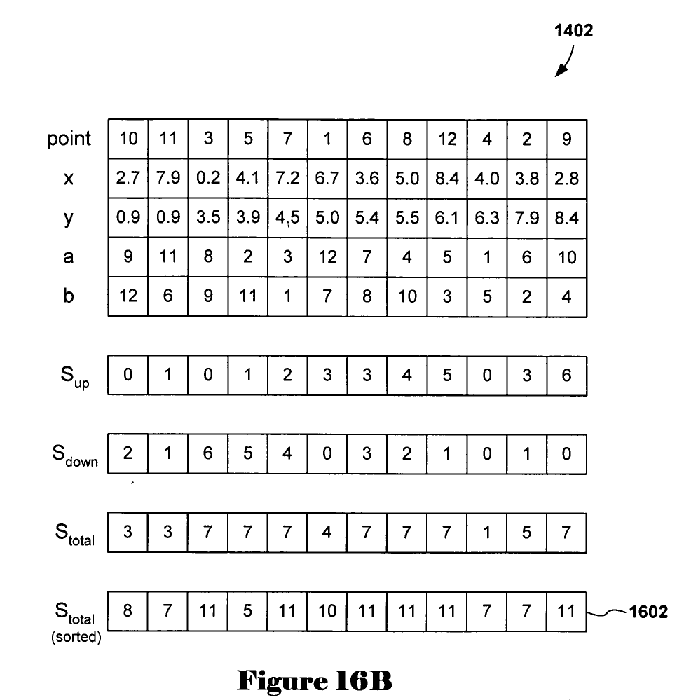

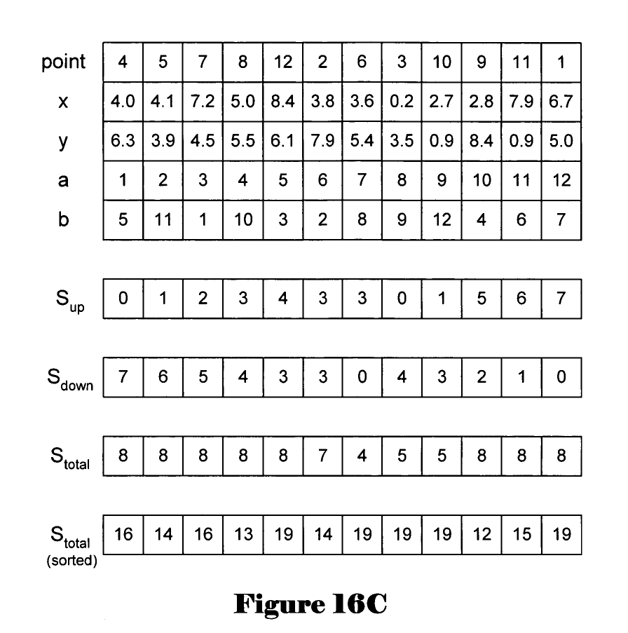

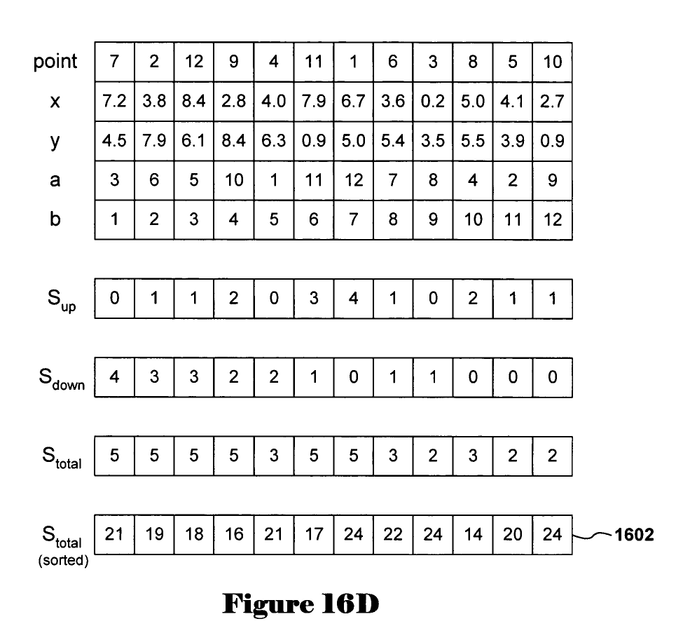

FIGS. 16A-D illustrate application of a more sophisticated method for producing an almost-LOPS set from the four-dimensional data-point distribution shown in FIG. 12. FIGS. 16A-D employ the same illustrative conventions as employed in FIGS. 14A-K. In this more sophisticated method, the data points are ordered, in successive steps, with respect to each of the four dimensions. The relaxed comparison operator illustrated in FIG. 15A is used, in each successive step, to traverse the five-row matrix first to the right, to generate Sᵤₚ values, and then to the left, to generate S_{down} values, resulting in Sₜₒₜₐₗ values for each step. The Sₜₒₜₐₗ values calculated in each step are cumulatively added to an S_{total(sorted)} array 1602 in which each element corresponds to the data point having the identity corresponding to the index of the data point. For example, the contents of S_{total(sorted)} element 1604, with index “1,” corresponds to the cumulative total for data point 1, represented in FIG. 16A by column 1606 in the five-row matrix 1402.

FIG. 16A shows the values resulting from a rightward and leftward traversal of the five-row matrix 1402 following ordering of the columns of the five-row matrix with respect to dimension x. Note that the values in the Sₜₒₜₐₗ array 1423 are not sorted with respect to data-point identity, but correspond to the data-point ordering in the five-row matrix 1402. Thus, each element in Sₜₒₜₐₗ is one greater than the sum of the corresponding elements of Sᵤₚ and S_{down}. However, the value “4” in element 1604 of the S_{total(sorted)} array 1602 corresponds to the value “4” in element 1606 of the Sₜₒₜₐₗ array 1423. FIG. 16B shows the values produced in the second step, following a rightward and leftward traversal of the five-row matrix 1402 ordered with respect to dimension y. FIG. 16C illustrates the values generated in a third step, following rightward and leftward traversals of the five-row matrix with columns ordered with respect to dimension a. FIG. 16D shows the values generated in a fourth step, following rightward and leftward traversal of the five-row matrix with columns ordered with respect to dimension b. The values in the Sₜₒₜₐₗ sorted array 1602 at the end of the fourth step represent almost-LOPS scores, by which an almost-LOPS data set can be selected. For example, one may decide to select data points with almost-LOPS values greater than the value “20” for the almost-LOPS set, resulting in selection of data points 1, 5, 7, 8, 9, and 12 which have corresponding almost-LOPS values of 21, 21, 24, 22, 24, and 24, respectively. Using a different threshold value produces LOPS sets with greater or fewer data-point members. Unlike in the case of LOPS or HOPS computation, the almost-LOPS or almost-HOPS computation does not necessarily lead to the maximally sized or maximally weighted order preserving sequence. However, the computation does lead to a near maximally sized or near maximally weighted order preserving sequence. By choosing almost-LOPS or almost-HOPS values greater than a threshold value, a reasonable set of normalizing data points is obtained.

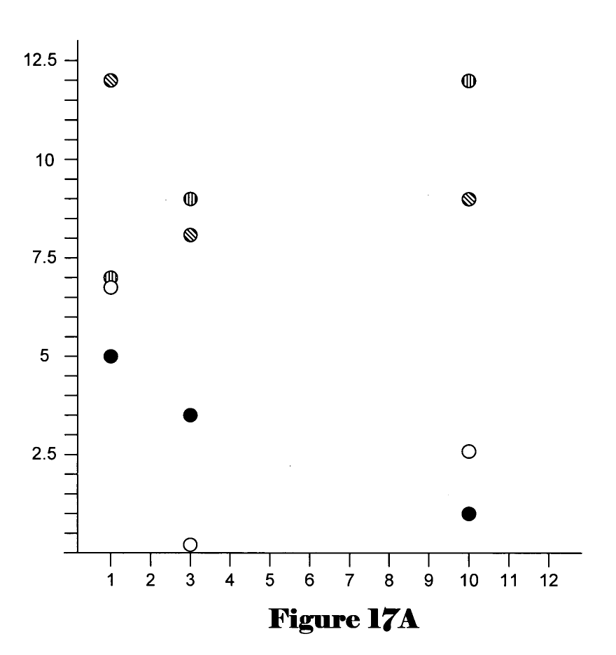

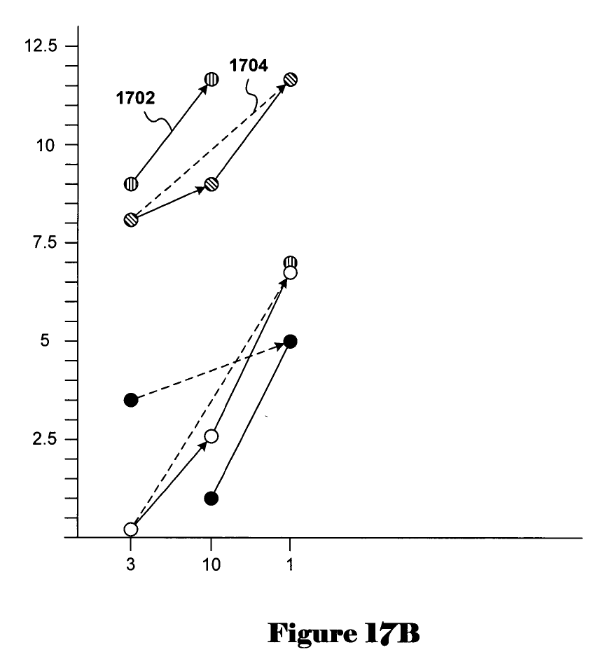

To illustrate the obtained results, displayed in FIG. 16D, the three data points with almost-LOPS values of 24, shown in the S_{total(sorted)} array 1602 in FIG. 16D, are considered. FIGS. 17A-B illustrate the three highest almost-LOPS data points graphed in the manner of FIG. 12. In FIG. 17A, the three data points, with data point identifiers “1,” “3,” and “10” are plotted alone in a graph similar to that of FIG. 12. In FIG. 17B, the three data points are plotted in the order “3,” “10,” and “1.” As can be seen by the positive-sloped solid arrows in FIG. 17B, such as solid arrow 1702, in three of the four dimensions, the values for data point “10” are greater than the values for data point “3,” and the values for data point “1” are greater than the values for data point “10.” The dashed arrows, such as dashed arrow 1704, show that, in three of the four dimensions, the values for data point “1” are greater than the values for data point “3.” Therefore, data points “3,” “10,” and “1” comprise an almost-LOPS, under the relaxed comparison operator described above with reference to FIG. 15A.

As mentioned above, without employing a sort step as a first step, the method may be slow. The sort provides a dimension different from all others in that it is truly order-preserved, or a “golden” dimension. However, there is generally no data set that can be considered more golden than the others, with the possible exception of a reference sample. To mitigate the effects of the asymmetry, a new “golden” dimension (N+1) can be added, where the new dimension is the rank of the sum of the ranks of all of the original dimensions. This new “golden” dimension then becomes the dimension for the sort.

The method treats the sample and the references as similar data, that we expect have similarly ordered expression levels. This is only the case for references comprised of pooled samples, or samples very similar to the biological sample being studied. An improvement that addresses this issue, for two-color or multicolor systems, is to independently compute LOPS scores for all the data in each channel. This improvement is particularly compelling in cases in which the reference is substantially different (biologically) from the unknown samples. This improvement greatly reduces the constraints and allows much longer sequences. In this embodiment, for n arrays, the n-dimensional LOPS score is calculated for each probe in each color independently, and the sum of the two sequence lengths is used as the total score for the probe. Then a LOPS tolerance is used to determine which probes to include in the normalization set, the LOPS-tolerance a parameter corresponding to the difference between the highest LOPS score and those of the points close to the highest score. The approach of summing scores can be generalized to summing scores of any set of subsets of the original dimensions. For example, the dimensions can be considered in pairs and the 2-dimensional LOPS for a larger number of pairs can be determined. Although the latter method may appear to be slow, this method can be implemented to run in order n*N log(N) in time, where N is the total number of points and n is the number of original dimensions.

### Multi-Dimensional HOPS

As noted above, the computational method for finding a HOPS or almost-HOPS is quite similar to the above-described method for finding a LOPS or almost-LOPS. Rather than provide a second, detailed example to illustrate the computational method for finding a HOPS or almost-HOPS, a brief, mathematical description of the HOPS-finding method is provided below.

For two vectors u and v in P^{D}, U≦v if, for all coordinates 1≦i≦D, uᵢ≦vᵢ. A set of points v(1), v(2), . . . , v(k) in P^{D} is an order preserving sequence if v(i)≦v(i+1), for all 1≦i≦k−1. Each point v(i) is associated with a weight s(i). The local heaviest order preserving sequence for a point v(i), where 1≦i≦k, is the order preserving sequence that includes point v(i) for which the sum of the weights associated with the points in the sequence is greatest. An O(N²) algorithm, linear in dimension D, for computing the local heaviest order preserving sequence for each point v(i), with coordinates v(i,1), . . . ,v(i,D), within a set of points v(1), v(2), . . . , v(k) in P^{D}, is next provided:

Input:

(a) a set of points v(1), v(2), . . . , v(N) in P^{D};
(b) the weights s(1), s(2), . . . , s(N) associated with the set of points v(1), v(2), . . . ,
   v(N)
      Output:
      For every index i, 1≦i≦N, the weight of the heaviest order preserving sequence that goes through the point v(i).
      Comment:
      The output of the algorithm can be more precisely stated as follows:
Denote by ω=ω(1), . . . , ω(λ) a subset of indices in 1 . . . N. The subset of indices ω is order preserving if v(ω(1)), . . . , v(ω(λ)) is order preserving.
For 1≦i≦N set

$$Ω ( i ) = \{ ω \text{:} ω is order preserving ⋀ i = ω ( j ) for some j \}$$

$$σ ( i ) = {max}_{ω ∈ Ω ( i )} ( {\sum }_{r = 1}^{λ ( ω )} s ( ω ( r ) ) )$$

and then output σ(i), 1≦i≦N.

The algorithm can be easily modified to output the sequences corresponding to each σ(i), or to simply output the sequence corresponding to maxᵢ(σ(i)).

Dynamic Programming Algorithm:.

1. Sort v(1), v(2), . . . , v(N) in v(i,1) ascending order: v(π1), v(π2), . . . , v(πN).
2. Set σ_{L}(π1)=s(π1), σ_{R}(πN)=s(πN).
3. For i=2 . . . N do
   σ_{L}(πᵢ)=s(πi)+max_{{j:v(πj)<v(πi)}}(σ_{L}(πj)).
4. For i=N−1 . . . 1 do
   σ_{R}(πi)=s(πi)+max_{{j:v(πj)>v(πi)}}(σ_{R}(πj))).
5. For i=1 . . . N do
   σ(i)=σ_{L}(i)+σ_{R}(i)−s(i).
      Comments:
(a) max(Ø)=0.
(b) When s(1)=1 for 1≦i≦N, LOPSs are found.
(c) The sequence corresponding to σ(i) can be determined by:
   (1) maintaining pointers associated with the σ_{L}(πi) and σ_{R}(πi);
   (2) modifying steps 3 and 4 to set the corresponding pointers to point (left or right)
      to the index that attains the maximum; and
   (3) modifying step 5 to collect the local HOPS at i by traversing the pointers.

As discussed above, the weights associated with points or vectors may reflect a confidence in the values of the points or vectors on an individual basis, based on statistical or other considerations, or on a dimension-by-dimension basis, based on statistical or other considerations related to entire data sets. HOPS is a superset of LOPS, essentially including an additional parameter for each data point and considering that parameter in determining an order preserving sequence.

### A C++-Like, Pseudocode Implementation of a Lops or Almost-LOPS Set Determination Method That Represents a Portion of One Embodiment of the Present Invention

In this subsection, a C++-like pseudocode implementation of a LOPS set or almost-LOPS set method is provided. In this implementation, the identities of points and indexes of the various arrays, described above with reference to FIGS. 14A-16D, start with the value “0” rather than the value “1.” Otherwise, this implementation faithfully follows the method described with respect to FIGS. 16A-D. Compiling and running this implementation produces the results shown in FIG. 16D.

First, the C++ pseudocode implementation includes various C-library include files defines the number of constants and types:
1 #include <stdio.h>
2 #include <stdarg.h>
3 #include <stdlib.h>
4 #include <string.h>
5 #include <stdio.h>
6 const int MAX_DATA=100;
7 const int MIN_DATA=5;
8 const int MAX_DIM=100;
9 const double BAD_VAL=−10000000;
10 const int BAD_VALUE=−10000000;
11 typedef bool COMP(double, double);
12 typedef COMP* COMP_PTR;
   The constant “MAX_DATA” is the maximum number of data points that can be included in each data set for the n-dimensional LOPS or almost-LOPS determination. The constant “MIN_DATA” is the smallest data set that can be accommodated. It simply makes no sense to determine LOPS or almost-LOPS sets for a tiny data set. The constant “MAX_DIM” is the maximum number of dimensions, or data sets, that can be accommodated. The constants “BAD_VAL” and “BAD_VALUE” are used to indicate error values. The type definition “COMP,” declared above on line 11, declares a type corresponding to a function that takes two arguments of type double and returns a Boolean value. This type definition is used to declare comparison functions that are passed by reference to the class “lopsSet,” described below. The type definition “COMP_PTR,” declared above on line 12, is a reference type that references a function of type “COMP.”

Next, a class “dataSet” is declared below:

| | |
|---|---|
| 1 | class dataSet |
| 2 | { |

| | |
|---|---|
| 3 | private: |

| | |
|---|---|
| 4 | double data[MAX_DATA]; |
| 5 | int sz; |

| | |
|---|---|
| 6 | public: |

| | |
|---|---|
| 7 | void clear( ) {sz = 0;};; |
| 8 | double operator [] (int i) {if (i < sz) return data[i]; |

| | |
|---|---|
| 9 | else return BAD_VAL;}; |

| | |
|---|---|
| 10 | double getVal(int i) {if (i < sz) return data[i]; |

| | |
|---|---|
| 11 | else return BAD_VAL;}; |

| | |
|---|---|
| 12 | void add(int num, double val ...); |
| 13 | int getSize( ) {return sz;}; |
| 14 | dataSet( ); |
| 15 | ˜dataSet( ); |

| | |
|---|---|
| 16 | }; |

An instance of the class “dataSet” is used to store the values of a data set. The data values, of type “double” are stored in the private data member “data,” declared above on line 4. The number of data points in the data set is stored in the integer data member “sz,” declared above on line 5. The class “dataSet” includes the following function members: (1) “clear,” declared above on line 7, that clears an instance of the class “dataSet” or, in other words, removes all values from the data set; (2) operator “[ ],” which returns the data value stored in the element of the private data member “data” at the index furnished by integer argument “i;” (3) “getVal,” declared above on line 10, which returns the value stored in the element of the private data member “data” with index equal to value of the argument “i;” (4) “add,” a function member that adds the series of values specified in the variable argument list beginning with argument “val” to the data set; (5) “getSize,” declared above on line 13, which returns the number of data points in the data set; and (6) a constructor and destructor for the class.

Rather straightforward implementations for the member function “add,” the constructor, and the destructor for class “dataSet” are provided below, without further annotation:

| | |
|---|---|
| 1 | void dataSet::add(int num, double val ...) |
| 2 | { |

| | |
|---|---|
| 3 | va_list ap; |
| 4 | double j; |
| 5 | va_start(ap, num); |
| 6 | while(num−− > 0) |
| 7 | { |

| | |
|---|---|
| 8 | j = va_arg(ap, double); |
| 9 | data[sz++] = j; |

| | |
|---|---|
| 10 | } |
| 11 | va_end(ap); |

| | |
|---|---|
| 12 | } |
| 1 | dataSet::dataSet( ) |
| 2 | { |

| | |
|---|---|
| 3 | sz = 0; |

| | |
|---|---|
| 4 | } |
| 1 | dataSet::˜dataSet( ) |
| 2 | { |
| 3 | |
| 4 | } |

The type definition “Dptr,” declared above on line 1, is a reference type, or pointer type, referring to an instance of the class “dataSet.” The type “OrderedD” declared above on lines 2-6, is a structure that contains the two elements “val” and “dex,” declared above on lines 4-5, that store a value and identity associated with a data point.

Next, the declaration of a class “lopsSet” is provided below:

| | |
|---|---|
| 1 | class lopsSet |
| 2 | { |

| | |
|---|---|
| 3 | private: |

| | |
|---|---|
| 4 | Dptr nDimensions[MAX_DIM]; |
| 5 | OrderedD S_order[MAX_DATA]; |
| 6 | int S_up[MAX_DATA]; |
| 7 | int S_down[MAX_DATA]; |
| 8 | int S_total[MAX_DATA]; |
| 9 | int lSet[MAX_DATA]; |
| 10 | bool setDone; |
| 11 | int lopsSetSz; |
| 12 | int numDim; |
| 13 | int dimSz; |
| 14 | int next; |
| 15 | COMP_PTR cmptr; |

| | |
|---|---|
| 16 | public: |

| 17 | void clear( ) {setDone = false; lopsSetSz = 0; |
|---|---|
| | numDim = 0;}; |
| 18 | void nextLops(int numD, COMP_PTR cmp, |

| | |
|---|---|
| 19 | int relax, int top, Dptr d ...); |

| | |
|---|---|
| 20 | void reorder(int cDim); |
| 21 | bool compar(int i, int j, int cDim, int relax); |
| 22 | int getSize( ) {return lopsSetSz;}; |
| 23 | int getFirstIndex( ); |
| 24 | int getNextIndex( ); |
| 25 | lopsSet( ); |
| 26 | ˜lopsSet( ); |

| | |
|---|---|
| 27 | }; |

The class “lopsSet” includes the following private data members: (1) “nDimensions,” declared above on line 4, an array containing references to all the data sets included in the LOPS or almost-LOPS analysis; (2) “S_order,” declared above on line 5, that contains the ordered totals contained in the array S_{total(sorted)} of FIGS. 16A-D, above; (3) integer arrays “S_up,” “S_down,” and “S_total,” declared above on lines 6-8, that correspond to the one-dimensional arrays Sup, S_{down}, and Sₜₒₜₐₗ in FIGS. 16A-D; (4) “1Set,” declared above on line 9, which contains the identities, or indices, of the points selected for the LOPS or almost-LOPS set; (5) “setDone,” declared above on line 10, which indicates whether or not the LOPS or almost-LOPS set has been calculated from the data sets referenced by the array “nDimensions,” described above; (6) “lopsSetSz,” declared above on line 11, which contains the number of data points in the LOPS or almost-LOPS set; (7) “numDim,” which contains the number of dimensions or data sets in which the LOPS set or almost-LOPS set is generated; (8) “dimSz,” declared above on line 13, which temporarily stores the number of data points in the data set corresponding to a particular dimension; (9) “next,” used to mark the next element of the LOPS set or almost-LOPS set for retrieval; and (10) “cmptr,” declared above on line 15, a pointer to the comparison operator to be used in conducting the rightward and leftward traversals of the data points during each step of the method, as described above with reference to FIG. 16A-D. The class “lopsSet” includes the following function members: (1) “clear,” which initializes the LOPS set or almost-LOPS set to have zero members; (2) “nextLops,” declared above on line 18, which determines the LOPS or almost-LOPS set from the data points of the data sets; (3) “reorder,” declared above on line 20, which reorders stretches of data points ordered with respect to one dimension that all have the same value in that dimension; (4) “compar,” declared above on line 21, which generally implements comparison operators of the types described above with reference to FIGS. 15A-B; (5) “getSize,” declared above on line 22, which returns to size of the LOPS set or almost-LOPS set; (6) “getFirstIndex” and “getNextIndex,” declared above on lines 23-24, which are used to retrieve the first and successive identities of data points within a LOPS or almost-LOPS set; and (7) a constructor and destructor. An instance of the class “lopsSet” therefore includes references to n data sets or dimension and the identities of the data points selected for a LOPS or almost-LOPS set via invocation of the function member “nextLops,” declared above on line 18. The arguments to function member nextLops include: (1) “numD,” the number of dimensions or data sets from which a LOPS set or almost-LOPS set is to be calculated, (2) “cmp,” a reference or pointer to a comparison operator that should be employed to compare corresponding values of data points during the rightward and leftward traversals of the set of data points; (3) “relax,” the number of dimensions to relax when applying the comparison operator during the rightward and leftward traversals; (4) “top,” the range of Sₜₒₜₐₗ ordered values, beginning with the highest value, to be used for selection of the LOPS set or almost-LOPS set; and (5) a variably sized list of references to the data sets to be included in the LOPS calculation.

An implementation of a comparison function, a reference to which may be supplied as argument “cmp” to lopsSet function member “nextLops,” is provided below:

| | |
|---|---|
| 1 | bool lessThan(double i, double j) |
| 2 | { |

| | |
|---|---|
| 3 | return (i < j); |

| | |
|---|---|
| 4 | } |

Implementation of a comparison function that can be supplied to the library routine “qsort” to sort the elements of the array “S_order” is next provided:

| | |
|---|---|
| 1 | int compare( const void* elem1, const void* elem2 ) |
| 2 | { |

| 3 | if (((OrderedD*)elem1)−>val < ((OrderedD*)elem2)−>val) |
|---|---|
| | return −1; |
| 4 | else if (((OrderedD*)elem1)−>val == |
| | ((OrderedD*)elem2)−>val) return 0; |
| 5 | else return 1; |

| | |
|---|---|
| 6 | } |

Next, an implementation of the lopsSet member function “nextLops” is provided:

| 1 | void lopsSet::nextLops(int numD, COMP_PTR cmp, int relax, |
|---|---|
| | int top, Dptr d ...) |
| 2 | { |

| | |
|---|---|
| 3 | va_list ap; |
| 4 | Dptr j; |
| 5 | int i, k, m, max; |
| 6 | clear( ); |
| 7 | cmptr = cmp; |
| 8 | va_start(ap, top); |
| 9 | while(numD−− > 0) |
| 10 | { |

| | |
|---|---|
| 11 | j = va_arg(ap, Dptr); |
| 12 | if (numDim == 0) |
| 13 | { |

| | |
|---|---|
| 14 | dimSz = j−>getSize( ); |
| 15 | if (dimSz < MIN_DATA) return; |

| | |
|---|---|
| 16 | } |
| 17 | else if (j−>getSize( ) != dimSz) return; |
| 18 | nDimensions[numDim++] = j; |

| | |
|---|---|
| 19 | } |
| 20 | va_end(ap); |
| 21 | if (numDim < 2) return; |
| 22 | for (i = 0; i < dimSz; i++) |

| | |
|---|---|
| 23 | S_total[i] = 0; |

| | |
|---|---|
| 24 | for (m = 0; m < numDim; m++) |
| 25 | { |

| | |
|---|---|
| 26 | for (i = 0; i < dimSz; i++) |
| 27 | { |

| | |
|---|---|
| 28 | S_order[i].val = nDimensions[m]−>getVal(i); |
| 29 | S_order[i].dex = i; |

| 30 | } |
|---|---|
| 31 | qsort( (void *)S_order, (size_t)dimSz, |
| | sizeof( OrderedD ), compare ); |
| 32 | reorder(m); |
| 33 | for (i = 0; i < dimSz; i++) |
| 34 | { |

| | |
|---|---|
| 35 | S_up[i] = 0; |
| 36 | S_down[i] = 0; |

| | |
|---|---|
| 37 | } |
| 38 | for (i = 1; i < dimSz; i++) |
| 39 | { |

| | |
|---|---|
| 40 | max = −1; |
| 41 | for (k = 0; k < i; k++) |

| 42 | if (compar (S_order[k].dex, |
|---|---|
| | S_order[i].dex, m, relax)) |

| | |
|---|---|
| 43 | if (max < S_up[k]) max = S_up[k]; |

| | |
|---|---|
| 44 | if (max > −1) S_up[i] = max + 1; |

| | |
|---|---|
| 45 | } |
| 46 | for (i = dimSz − 2; i >= 0; i−−) |
| 47 | { |

| | |
|---|---|
| 48 | max = −1; |
| 49 | for (k = dimSz − 1; k > i; k−−) |

| 50 | if (compar (S_order[i].dex, |
|---|---|
| | S_order[k].dex, m, relax)) |

| | |
|---|---|
| 51 | if (max < S_down[k]) max = S_down[k]; |

| | |
|---|---|
| 52 | if(max > −1) S_down[i] = max + 1; |

| | |
|---|---|
| 53 | } |
| 54 | for (i = 0; i < dimSz; i++) |

| 55 | S_total[S_order[i].dex] += |
|---|---|
| | S_up[i] + S_down[i] + 1; |

| | |
|---|---|
| 56 | } |
| 57 | max = −1; |
| 58 | for (i = 0; i < dimSz; i++) |
| 59 | { |

| | |
|---|---|
| 60 | if (S_total[i] > max) max = S_total[i]; |

| | |
|---|---|
| 61 | } |
| 62 | for (i = 0; i < dimSz; i++) |
| 63 | { |

| | |
|---|---|
| 64 | if (S_total[i] >= max − top) |

| | |
|---|---|
| 65 | lSet[lopsSetSz++] = i; |

| | |
|---|---|
| 66 | } |
| 67 | setDone = true; |

| | |
|---|---|
| 68 | } |

First, the variables described in the LOPS set are cleared via a call to function member “clear” on line 6. Next, the private data member “cmptr” is initialized on line 7. In the while-loop of lines 9-19, references to instances of the class “dataSet” are collected from the variable argument list and placed into the private member array “nDimensions.” Note that all the data sets must be of the same size, or the function member returns. In practicality, data points selected for normalization should be present in each of the data sets considered. Of course, this restriction might be relaxed in alternative implementations. In the for-loop of lines 22-23, the array S_total is initialized to contain the value “0” in each element. Next, each step of the method, described in the example above with reference to each of FIGS. 16A-D, is carried out in the for-loop of lines 24-56. Note that the for loop iterates over each data set, the for-loop variable “m” containing the index of the data set within the private data member “nDimensions.” In each step, the array “S_order” is initialized in the for-loop of lines 26-30. Then, on lines 31-32, the elements of the array “S_order” are sorted via routines “qsort” and “reorder.” Following sorting, the array “S_order” contains the values of the data points for the dimension currently being sorted, or considered, in the current step, paired with the identities of the corresponding data points, which are the indices of the data points within the instance of the class “dataSet” in which they are stored. Note that the function “reorder” represents an optional step in which sub-sequences of data points within the set of data points sorted with respect to values in the currently considered dimension may be reordered based on consideration of additional dimensions. In the for-loop of lines 33-37, the elements of the arrays “S_up” and “S_down” are set to contain the value “0.” In the for-loop of lines 38-45, the rightward traversal of the ordered set of data points is carried out, and in the for-loop of lines 46-53, the leftward traversal of the ordered set of data points is carried out. Following execution of these two for-loops, the arrays “S_up” and “S_down” are filled with values for each data point. Next, in the for-loop of lines 54-55, the values in the S_up and S_down arrays are added together and incremented, and these values are added to the cumulative totals stored in the array “S_total.” Following completion of the for-loop of lines 24-56, the LOPS score for each data point has been calculated and stored in the array “S_total.” Then, the maximum LOPS score is determined in the for-loop of lines 58-61, and the LOPS set or almost-LOPS set obtained by execution by the for-loop of lines 62-66.

Next, an implementation for the optional function member “reorder” is provided, without further annotation:

| | |
|---|---|
| 1 | void lopsSet::reorder(int cDim) |
| 2 | { |

| | |
|---|---|
| 3 | int i, j, k, l; |
| 4 | bool changed; |
| 5 | OrderedD T; |
| 6 | int relaxable = (numDim / 2) − 1; |
| 7 | for (i = 0; i < dimSz − 1;) |
| 8 | { |

| 9 | for (j = i; S_order[j].val == |
|---|---|
| | S_order[i].val && j < dimSz; j++); |
| 10 | j−−; |
| 11 | if (j > i) |

| | |
|---|---|
| 12 | do |
| 13 | { |

| | |
|---|---|
| 14 | changed = false; |
| 15 | for (k = i, l = i+ 1; |

l <= j; k++, l++)

| 16 | if (compar(S_order[l].dex, |
|---|---|
| | S_order[k].dex, cDim, relaxable)) |
| 17 | { |

| | |
|---|---|
| 18 | changed = true; |
| 19 | T = S_order[k]; |
| 20 | S_order[k] = S_order[l]; |
| 21 | S_order[l] = T; |

| | |
|---|---|
| 22 | } |

| | |
|---|---|
| 23 | } |
| 24 | while (changed); |

| | |
|---|---|
| 25 | i = j + 1; |

| | |
|---|---|
| 26 | } |

| | |
|---|---|
| 27 | } |

Next, an implementation of the comparison operator which carries out a comparison of the type described with reference to FIGS. 15A-B is provided:

| | |
|---|---|
| 1 | bool lopsSet::compar(int i, int j, int cDim, int relax) |
| 2 | { |

| | |
|---|---|
| 3 | int k; |
| 4 | int numTrue = 0; |
| 5 | for (k = 0; k < numDim; k++) |
| 6 | { |

| | |
|---|---|
| 7 | if (k == cDim) continue; |
| 8 | if ((*cmptr)(nDimensions[k]−>getVal(i), |

nDimensions[k]−>getVal(j)))

| | |
|---|---|
| 9 | numTrue++; |

| | |
|---|---|
| 10 | } |
| 11 | return (numTrue >= (numDim − relax − 1)); |

| | |
|---|---|
| 12 | } |

The function member “compar” simply iterates through all the dimensions, and totals the number of TRUE values obtained in pair-wise comparisons in the for-loop of lines 5-10. Then, in line 11, the function member “compar” determines the final Boolean value for the comparison operator which depends on the number of TRUE values detected and on the number of dimensions that are relaxed.

Next, elimination of functions that allow for retrieval of the identities of the LOPS set or almost-LOPS set are provided, along with a constructor and destructor for the class “lopsSet,” without further annotation:

| | |
|---|---|
| 1 | int lopsSet::getFirstIndex( ) |
| 2 | { |

| | |
|---|---|
| 3 | if (setDone) |
| 4 | { |

| | |
|---|---|
| 5 | if (lopsSetSz > 0) |
| 6 | { |

| | |
|---|---|
| 7 | next = 1; |
| 8 | return lSet[0]; |

| | |
|---|---|
| 9 | } |
| 10 | else return BAD_VALUE; |

| | |
|---|---|
| 11 | } |
| 12 | return BAD_VALUE; |

| | |
|---|---|
| 13 | } |
| 1 | int lopsSet::getNextIndex( ) |
| 2 | { |

| | |
|---|---|
| 3 | if (next < lopsSetSz) return lSet[next++]; |
| 4 | else return BAD_VALUE; |

| | |
|---|---|
| 5 | } |
| 1 | lopsSet::lopsSet( ) |
| 2 | { |

| | |
|---|---|
| 3 | clear( ); |

| | |
|---|---|
| 4 | } |
| 1 | lopsSet::˜lopsSet( ) |
| 2 | { |
| 3 | |
| 4 | } |

Finally, a simple function “main,” which employs instances of the class “dataSet” and “lopsSet” to compute the almost-LOPS set computed in the example of FIG. 16A-D, is provided:

| | |
|---|---|
| 1 | int main(int argc, char* argv[]) |
| 2 | { |

| 3 | dataSet d1; |
|---|---|
| 4 | dataSet d2; |
| 5 | dataSet d3; |
| 6 | dataSet d4; |
| 7 | lopsSet lops; |
| 8 | int i; |
| 9 | d1.add(12, 6.7, 3.8, 0.2, 4.0, 4.1, 3.6, 7.2, 5.0, 2.8, |
| | 2.7, 7.9, 8.4); |
| 10 | d2.add(12, 5.0, 7.9, 3.5, 6.3, 3.9, 5.4, 4.5, 5.5, 8.4, |
| | 0.9, 0.9, 6.1); |
| 11 | d3.add(12, 12.0, 6.0, 8.0, 1.0, 2.0, 7.0, 3.0, 4.0, 10.0, |
| | 9.0, 11.0, 5.0); |
| 12 | d4.add(12, 7.0, 2.0, 9.0, 5.0, 11.0, 8.0, 1.0, 10.0, 4.0, |
| | 12.0, 6.0, 3.0); |
| 13 | lops.nextLops(4, lessThan, 1, 3, &d1, &d2, &d3, &d4); |
| 14 | return 0; |

| | |
|---|---|
| 15 | } |

Although the present invention has been described in terms of a particular embodiment, it is not intended that the invention be limited to this embodiment. Modifications within the spirit of the invention will be apparent to those skilled in the art. For example, an almost limitless number of different implementations may be devised to carry out selection of LOPS or almost-LOPS sets from a number of data sets. As discussed, a large variety of different comparison operators may be devised for the traversals of the data points, and various different types of constraint relaxation may be employed. Additional manipulations of the basic method are possible. Distributions may be seeded with artificial LOPS-set data points, to ensure reasonable behavior at the extremes of LOPS sequences. LOPS sets may be additionally selected based on entropy, or by applying additional constraints, including selecting LOPS sequences with particular geometries or Euclidean dimensions. As discussed above, the method described above can be used to calculate HOPS, rather than LOPS, from n-dimensional data-point distributions. The values placed into the Sᵤₚ and S_{down} arrays are the weighted value of the currently considered data point added to the maximum value in the Sᵤₚ and S_{down} subsequences to the right and left of the currently considered data point, and the final Sₜₒₜₐₗ values are obtained by subtracting the weighted value of a data point from the sum of the entries in the Sᵤₚ and S_{down} arrays for the data point. Although the method of the present invention has been discussed using microarray data examples, it is equally applicable to general data-set normalization problems where the data is obtained from a variety of sources. There are a large variety of alternative, LOPS-like computational methods that represent alternative embodiments of the present invention. These alternative methods include: (1) LOPS incidence; (2) shortest-Euclidean LOPS; and LOPS even partitioning. LOPS incidence involves carrying out a number of 2-diemsnional LOPS computations for red/green two-channel arrays, for normalizing the red signals to the green signals, and then selecting as global normalization features those features that most frequently appear in the computed 2-dimensional LOPS, or that occur with a frequency greater than a threshold frequency. Shortest-Euclidean LOPS involves selecting a particular LOPS from among a number of LOPS of equal or approximately equal length, the selected LOPS having the shortest sum of Euclidean, linear distances between the features of all of the equal, or approximately equal, length LOPS. LOPS even partitioning involves selecting LOPS features that as evenly as possible partition the data set into subsets of features with signal-intensity values between adjacent LOPS features. More elaborate, statistically selected LOPS points may be used, and various combinations of procedures and relaxed comparison operators may be used to select normalization features among the features having computed LOPS values greater than a threshold value. In certain cases, only a subset of the available dimensions may be employed to compute LOPS, HOPS, almost-LOPS, or almost LOPS sequences, with the choice dependent on additional information about the quality or reliability of the data sets.

The foregoing description, for purposes of explanation, used specific nomenclature to provide a thorough understanding of the invention. However, it will be apparent to one skilled in the art that the specific details are not required in order to practice the invention. The foregoing descriptions of specific embodiments of the present invention are presented for purpose of illustration and description. They are not intended to be exhaustive or to limit the invention to the precise forms disclosed. Obviously many modifications and variations are possible in view of the above teachings. The embodiments are shown and described in order to best explain the principles of the invention and its practical applications, to thereby enable others skilled in the art to best utilize the invention and various embodiments with various modifications as are suited to the particular use contemplated. It is intended that the scope of the invention be defined by the following claims and their equivalents:

1. A method for selecting a set of normalizing data points from n data sets, where n is at least 3, containing data points having values and identities, the method comprising:
receiving n data sets;
considering the data points to be distributed in an n-dimensional data-point space;
determining one or more order-preserving sequences of data points within the n-dimensional data-point space; and
selecting, as normalizing data points, data points from the one or more order-preserving sequences.

2. The method of claim 1 wherein the one or more order-preserving sequences of data points is a single, longest order-preserving sequence of data points.

3. The method of claim 1 wherein the data points within n data sets are associated with weights and wherein the one or more order-preserving sequences of data points is an order-preserving sequence of data points with a greatest sum of weights.

4. The method of claim 1 wherein the one or more order-preserving sequences of data points is a longest order-preserving sequence of data points having a shortest Euclidian distance accumulated along a path from an initial data point of the order-preserving sequence to a final data point of the order-preserving sequence.

5. The method of claim 1 wherein the one or more order-preserving sequences of data points are order-preserving sequences of data points of lengths within a threshold value of the length of an order-preserving sequence of data points of maximum length.

6. The method of claim 1 wherein the data points within n data sets are associated with weights and wherein the one or more order-preserving sequences of data points are order-preserving sequences of data points with sums of weights within a threshold value of the sum of weights of an order-preserving sequence of data points with a greatest sum of weights.

7. The method of claim 1 wherein considering the data points to be distributed in an n-dimensional data-point space further includes, for each data point, considering the data point to have a value in each of n-dimensions, the value of a data-point in an ith dimension equal to the value of the data point in an ith data set, where 1≦i≦n.

8. The method of claim 1 wherein determining an order-preserving sequence of data points within the n-dimensional data-point space further includes:
for each currently considered dimension,
ordering the data points with respect to the currently considered dimension;
traversing the ordered data points in a first direction, determining a metric corresponding to a maximum subsequence for each data point in the first direction; and
traversing the ordered data points in a second direction, determining a metric corresponding to a maximum subsequence for each data point in the second direction;
summing the determined metrics for each data point in each dimension to produce a metric sum for each data point; and
selecting as belonging to the maximum order-preserving sequence of data points those data points having a greatest metric sum.

9. The method of claim 8 wherein selecting, as normalizing data points, data points from the order-preserving sequence further includes selecting data points with a metric sum greater than a threshold value.

10. The method of claim 8 wherein selecting, as normalizing data points, data points from the one or more order-preserving sequences further includes selecting data points of a single order-preserving sequence.

11. The method of claim 8 wherein selecting, as normalizing data points, data points from the one or more order-preserving sequences further includes selecting data points that most evenly partition the data points into subsets of data points.

12. Computer instructions stored in a computer readable medium that implement the method of claim 1.

13. A data set normalized according to the method of claim 1 stored in a computer readable medium.

14. A system for selecting a set of normalizing data points from n data sets, where n is at least 3, containing data points having values and identities, the system comprising:
a processor;
a memory;
and computer instructions that select the set of normalizing data points from n data sets by
receiving n data sets,
considering the data points to be distributed in an n-dimensional data-point space,
determining one or more order-preserving sequence of data points within the n-dimensional data-point space, and
selecting, as normalizing data points, data points from the one or more order-preserving sequences.

15. The method of claim 14 wherein the one or more order-preserving sequences of data points is a single, longest order-preserving sequence of data points.

16. The method of claim 14 wherein the data points within n data sets are associated with weights and wherein the one or more order-preserving sequences of data points is an order-preserving sequence of data points with a greatest sum of weights.

17. The method of claim 14 wherein the one or more order-preserving sequences of data points is a longest order-preserving sequence of data points having a shortest Euclidian distance accumulated along a path from an initial data point of the order-preserving sequence to a final data point of the order-preserving sequence.

18. The method of claim 14 wherein the one or more order-preserving sequences of data points are order-preserving sequence of data points within a threshold value of an order-preserving sequences of data points of maximum length.

19. The method of claim 14 wherein the one or more order-preserving sequences of data points are order-preserving sequence of data points within a threshold value of an order-preserving sequences of data points with a greatest sum of weights.

20. A method for selecting a set of normalizing data points from n data sets, where n is at least 4 and even, containing data points having values and identities, the method comprising:
receiving n data sets;
considering the data points to be distributed in n/2 2-dimensional data-point spaces;
determining one or more order-preserving sequences of data points for each of the n/2 2-dimensional data-point spaces; and
selecting, as normalizing data points, data points from the order-preserving sequences.

21. The method of claim 20 wherein the one or more order-preserving sequences of data points is a single, longest order-preserving sequence of data points.

22. The method of claim 20 wherein the data points within n data sets are associated with weights and wherein the one or more order-preserving sequences of data points is an order-preserving sequence of data points with a greatest sum of weights.

23. The method of claim 20 wherein the one or more order-preserving sequences of data points is a longest order-preserving sequence of data points having a shortest Euclidian distance accumulated along a path from an initial data point of the order-preserving sequence to a final data point of the order-preserving sequence.

24. The method of claim 20 wherein the one or more order-preserving sequences of data points are order-preserving sequences of data points within a threshold value of an order-preserving sequence of data points of maximum length.

25. The method of claim 20 wherein the data points within n data sets are associated with weights and wherein the one or more order-preserving sequences of data points are order-preserving sequences of data points with sums of weights within a threshold value of the sum of weights of an order-preserving sequence of data points with a greatest sum of weights.

26. The method of claim 20 wherein determining an order-preserving sequence of data points within a 2-dimensional data-point space further includes:
for each currently considered dimension,
ordering the data points with respect to the currently considered dimension;
traversing the ordered data points in a first direction, determining a metric corresponding to a maximum subsequence for each data point in the first direction; and
traversing the ordered data points in a second direction, determining a metric corresponding to a maximum subsequence for each data point in the second direction;
summing the determined metrics for each data point in each dimension to produce a metric sum for each data point; and
selecting as belonging to the maximum order-preserving sequence of data points those data points having a greatest metric sum.

27. The method of claim 20 wherein selecting, as normalizing data points, data points from the one or more order-preserving sequences further includes selecting data points which occur in the one or order-preserving sequences computed for greater than a threshold fraction of the n/2 2-dimensional data-point spaces.

28. Computer instructions stored in a computer readable medium that implement the method of claim 20.

29. A data set normalized according to the method of claim 20 stored in a computer readable medium.
